(19) United States
(12) Patent Application Publication
(10) Pub. No.: US 2016/0184165 A1
(43) Pub. Date: Jun. 30, 2016

(54) SWINGING JOINT DEVICE, WALKING-ABILITY ASSISTING DEVICE, AND METHOD FOR CONTROLLING RIGIDITY OF SWINGING JOINT

(71) Applicant: JTEKT CORPORATION, Osaka-shi (JP)

(72) Inventors: Hiromichi OHTA, Kariya-shi (JP); Kazuyoshi OHTSUBO, Chiryu-shi (JP); Yoshitaka YOSHIMI, Okazaki-shi (JP)

(73) Assignee: JTEKT CORPORATION, Osaka-shi (JP)

(21) Appl. No.: 14/979,702

(22) Filed: Dec. 28, 2015

(30) Foreign Application Priority Data
Dec. 24, 2014 (JP) 2014-260908
Dec. 24, 2014 (JP) 2014-260909
Dec. 24, 2014 (JP) 2014-260910
Oct. 15, 2015 (JP) 2015-203913

(51) Int. Cl. A61H 3/00; A61F 2/68

(52) U.S. Cl. CPC A61H 3/00; A61F 2/68

(57) ABSTRACT

A swinging joint device includes: a driving shaft member; a first swinging arm that is swingably supported about the driving shaft member; a driven shaft member that is arranged parallel to the driving shaft member; an interlocking swinging member that swings about the driven shaft member in an interlocking manner with swinging of the first swinging arm; an elastic body that is connected to the interlocking swinging member to generate an urging force in a direction opposite to an interlocking swinging direction of the interlocking swinging member; a rigidity variable portion that varies rigidity of the elastic body seen from the interlocking swinging member; a first angle detection portion that detects a swinging angle; and a control portion that controls the rigidity variable portion according to the swinging angle detected by the first angle detection portion to adjust the rigidity of the elastic body seen from the interlocking swinging member.

### INCORPORATION BY REFERENCE

The disclosure of Japanese Patent Applications No. 2014-260908, No. 2014-260909, and No. 2014-260910 filed on Dec. 24, 2014 and No. 2015-203913 filed on Oct. 15, 2015 each including the specification, drawings and abstract is incorporated herein by reference in its entirety.

### BACKGROUND OF THE INVENTION

1. Field of the Invention

The invention relates to a swinging joint device that performs cyclic swinging motion and varies the rigidity of a joint, a walking-ability assisting device that performs cyclic swinging motion to assist user's walking or running, and a method for controlling the rigidity of a swinging joint by which the rigidity of the joint that performs cyclic swinging motion is varied.

2. Description of Related Art

As an example of a device for controlling a joint that performs cyclic swinging motion, Japanese Patent Application Publication No. 2004-344304 (JP 2004-344304 A) discloses a walking assisting device that applies an assisting force to the lower limb (ranging from the hip joint to the foot) of a user. The walking assisting device has a waist-part outfit attached so as to wind the waist part of a user, a joining bar extending from the lateral side of the hip joint to the lateral side of the knee joint of the user, a crus-part outfit extending from the lateral side of the knee joint to the calf of the user, a hip joint actuator attached at the lateral position of the hip joint of the joining bar, and a knee joint actuator attached at the lateral position of the knee joint of the joining bar. Then, the hip joint actuator is attached to the joining portion of the waist-part outfit and swings the joining bar back and forth about the hip joint relative to the waist-part outfit on the lateral side of the hip joint. In addition, the knee joint actuator swings the crus-part outfit back and forth about the knee joint relative to the joining bar on the lateral side of the knee joint. Moreover, the hip joint actuator and the knee joint actuator are electric motors, and power is supplied to the electric motors from a battery attached to the waist-part outfit.

In addition, Japanese Patent Application Publication No. 2012-125388 (JP 2012-125388 A) discloses a walking rehabilitation device that assists the swinging motion of the crus (ranging from the knee to the ankle) of a user. The walking rehabilitation device has a controller arranged around the waist of a user, a femoral link extending from the lateral side of the hip joint to the lateral side of the knee joint of the user, crus links extending from both lateral sides of the knee joint to the ankle joint of the user, a motor arranged on the lateral side of the knee joint, and foot links extending from the ankle joint to the sole of the user. The motor is attached at the joining portion between the femoral link and the crus links and on the lateral side of the knee joint, and swings the crus links back and forth about the knee joint relative to the femoral link on the lateral side of the knee joint. Power is supplied to the motor from a battery included in the controller.

Moreover, Japanese Patent Application Publication No. 2013-236741 (JP 2013-236741 A) discloses a one-leg walking assisting machine that is attached to a leg in trouble of a user, of which one leg is in good condition and the other leg is in trouble, to assist the swinging motion of the leg in trouble. The one-leg walking assisting machine has a waist attachment portion arranged on the lateral side of the waist of a user, a femoral link portion extending from the lateral side of the hip joint to the lateral side of the knee joint of the user, a crus link portion extending downward from the lateral side of the knee joint, a torque generation unit arranged on the lateral side of the hip joint, and a damper arranged on the lateral side of the knee joint. The torque generation unit is constituted by a cam and a compression spring, generates a torque when a leg in trouble swings backward with the swinging of a leg in good condition, assists the swinging of the leg in trouble using the generated torque, and requires no actuator such as an electric motor. In addition, the torque generation unit is configured to be capable of adjusting an initial compression amount of the compression spring and varies a degree of a generated torque.

### SUMMARY OF THE INVENTION

Both the walking assisting device described in JP 2004-344304 A and the walking rehabilitation device described in JP 2012-125388 A assist the walking motion of a lower limb or a part of the lower limb with the electric motors but may not assist the walking motion when power is not continuously supplied from the batteries. In addition, since a user who requires walking assistance does not afford to carry a large and heavy battery, it is assumed that the batteries used in the above devices are relatively small and lightweight. In addition, JP 2004-344304 A and JP 2012-125388 A do not describe any specific configuration that reduces the consumption power of the electric motors. Accordingly, it is assumed that the continuous operation times of the assisting devices described in JP 2004-344304 A and JP 2012-125388 A are relatively short.

Moreover, the one-leg walking assisting machine described in JP 2013-236741 A generates a torque for swinging a leg through the cam and the compression spring without using an electric motor, and the continuous operation time of the assisting machine is longer than those of the assisting devices described in JP 2004-344304 A and JP 2012-125388 A. However, in order to correspond to a difference in body type (difference in inertia moment of a lower limb) for each user, a difference in swinging angle of a lower limb for each user, a user's physical condition, a difference in inclination of a walking place, or the like, it is required for a user to adjust the position of a determination portion provided on the compression spring of the torque generation unit with a tool such as a slotted screw driver and adjust an initial compression amount of the compression spring by hand. Therefore, such an operation becomes troublesome for the user.

The invention provides a swinging joint device, a walking-ability assisting device, and a method for controlling the rigidity of a swinging joint in which the rigidity of a joint performing swinging motion is automatically adjusted to be capable of automatically adjusting a torque generated by the swinging motion and further reducing consumption power or a user's load.

According to a first aspect of the invention, there is provided a swinging joint device including: a driving shaft member; a first swinging arm that is swingably supported about the driving shaft member; a driven shaft member that is arranged parallel to the driving shaft member; an interlocking swinging member that is connected to the first swinging arm via a power transmission portion to swing about the driven shaft member in an interlocking manner with swinging of the first swinging arm while swinging at an interlocking swinging angle smaller than a first swinging angle that is a swinging angle of the first swinging arm; an elastic body that is connected to the interlocking swinging member to generate an urging force corresponding to the interlocking swinging angle, the urging force being generated in a direction opposite to an interlocking swinging direction of the interlocking swinging member; a rigidity variable portion that varies rigidity of the elastic body seen from the interlocking swinging member; a first angle detection portion that detects one of the first swinging angle and the interlocking swinging angle; and a control portion that controls the rigidity variable portion according to one of the first swinging angle and the interlocking swinging angle detected by the first angle detection portion to adjust the rigidity of the elastic body seen from the interlocking swinging member.

According to the above first aspect, an apparent spring constant variable portion is controlled according to a first swinging angle or an interlocking swinging angle using the control portion. Therefore, since a degree of a torque required for assisting swinging motion is automatically adjusted for the swinging motion of a swinging object including a swinging arm, the torque may be adjusted without any trouble. In addition, since a torque required for assisting swinging motion is generated using an expansion/contraction spring, consumption power or a user's load may be further reduced.

In addition, in the above aspect, the elastic body may be an expansion/contraction spring, and the rigidity variable portion may be an apparent spring constant variable portion that varies an apparent spring constant of the expansion/contraction spring seen from the interlocking swinging member.

According to the above configuration, since the use of the expansion/contraction spring as the elastic body makes it possible to secure an optimum energy reservation amount and easily adjust a spring constant (rigidity) for a user's action such as walking and running, energy may be smoothly reserved and output.

In the above configuration, the apparent spring constant variable portion may be constituted by a rigidity adjustment shaft member that is arranged at a position near a periphery of the interlocking swinging member and arranged parallel to the driven shaft member, a rigidity adjustment shaft pivoting portion that pivots the rigidity adjustment shaft member, a pivoting member that is connected to the rigidity adjustment shaft member to pivot with the rigidity adjustment shaft member, and the expansion/contraction spring, a portion corresponding to a first end of the expansion/contraction spring may be connected to a spring fixing end of the pivoting member that is at a position away from the rigidity adjustment shaft member, a portion corresponding to a second end of the expansion/contraction spring may be connected to a spring swinging end that is at a position near the periphery of the interlocking swinging member, the spring swinging end being coaxial with the rigidity adjustment shaft member at the position when the interlocking swinging angle is zero, the expansion/contraction spring connected to the spring fixing end and the spring swinging end may have a free length when the interlocking swinging angle is zero, and the control portion may adjust a rigidity adjustment angle according to the interlocking swinging angle to adjust the apparent spring constant of the expansion/contraction spring seen from the interlocking swinging member, the rigidity adjustment angle being an angle formed between a virtual tangential line and a virtual line, the virtual tangential line representing a tangential line that is set on a circumference of a virtual interlocking swinging circle serving as a circle having a distance between the driven shaft member and the rigidity adjustment shaft member as a radius about the driven shaft member and that is set at a position of the rigidity adjustment shaft member, the virtual line connecting the spring swinging end and the spring fixing end to each other when the interlocking swinging angle is zero.

According to the above configuration, the apparent spring constant variable portion including the expansion/contraction spring may be specifically realized. In addition, since an apparent spring constant may be adjusted only by controlling the rigidity adjustment shaft portion with the control portion and pivoting the pivoting member, the apparent spring constant may be easily adjusted.

In the above configuration, two apparent spring constant variable portions may be attached to the interlocking swinging member as the apparent spring constant variable portion.

According to the above configuration, even when the expansion/contraction springs are, for example, springs that generate an urging force only in their expansion directions, the expansion/contraction spring of one the apparent spring constant variable portion may be configured to expand in its expansion direction relative to swinging motion in one direction and the expansion/contraction spring of the other apparent spring constant variable portion may be configured to expand in its expansion direction relative to swinging motion in the other direction. Therefore, the structures of the apparent spring constant variable portions may be further simplified.

In the above configuration, a first one of the two apparent spring constant variable portions attached to the interlocking swinging member may have the rigidity adjustment shaft pivoting portion, and a second one of the two apparent spring constant variable portions attached to the interlocking swinging member may not have the rigidity adjustment shaft pivoting portion but may have a pivoting member power transmission portion that transmits, to the pivoting member of the second apparent spring constant variable portion, a pivoting driving force of the pivoting member of the first apparent spring constant variable portion generated by the rigidity adjustment shaft pivoting portion of the first apparent spring constant variable portion.

According to the above configuration, since the two pivoting members may be pivoted at the same time by the one rigidity adjustment shaft pivoting portion, the structure may be further simplified.

In the above aspect, the swinging joint device may further include a first driving portion that swings the first swinging arm about the driving shaft member based on a control signal from the control portion.

According to the above configuration, the first driving portion swings the first swinging arm. Therefore, when the swinging joint device is used as, for example, a walking-ability assisting device that supports user's walking or running, a load may be further reduced when a user runs or walks.

In the above aspect, the swinging joint device may further include: a second swinging arm that is swingably supported about the driving shaft member; a second angle detection portion that detects a second swinging angle as a swinging angle of the second swinging arm; a second driving portion that swings the second swinging arm about the driving shaft member based on a control signal from the control portion; and a swinging link member that is connected to the first swinging arm and the second swinging arm to operate based on the first swinging angle of the first swinging arm and the second swinging angle of the second swinging arm.

According to the above configuration, when the swinging joint device is used as, for example, a walking-ability assisting device that supports user's walking or running, the first swinging arm may support the motion of the femoral part of a user and the second swinging arm may assist the crus part of the user. Therefore, a load may be further reduced when the user walks or runs.

In the above aspect, the power transmission portion that transmits swinging of the first swinging arm to the interlocking swinging member may be constituted by one of a gear, a belt, and a link mechanism.

According to the above configuration, the interlocking swinging member may appropriately swing in an interlocking manner when the swinging motion of the first swinging arm is appropriately transmitted to the interlocking swinging member.

According to a second aspect of the invention, there is provided a walking-ability assisting device for applying an assisting force to motion of a lower limb, the device including: a waist-side attachment portion that is attached to a waist-side part; a first longitudinal swinging arm that is arranged on a lateral side of a femur and has a shaft hole near an upper end thereof; a femoral attachment portion that is attached to the first swinging arm and put on the femur; a driving shaft member that is inserted into the shaft hole of the first swinging arm to swingably support the first swinging arm back and forth relative to the waist-side attachment portion; a rigidity variable portion that varies rigidity about the driving shaft member; and a control portion that controls the rigidity about the driving shaft member varied by the rigidity variable portion.

According to the above aspect, the rigidity variable portion is controlled using the control portion to control rigidity about the driving shaft member. Therefore, since a degree of a torque required for assisting swinging motion is automatically adjusted for the swinging motion of a swinging object including the first swinging arm, the torque may be adjusted without any trouble. In addition, since a torque required for assisting swinging motion is generated, consumption power or a user's load may be further reduced.

In the above aspect, the rigidity variable portion may have an expansion/contraction spring, the expansion/contraction spring may have a free length when a swinging angle of the first swinging arm is zero, and an expansion/contraction amount of the expansion/contraction spring may be varied relative to the swinging angle of the first swinging arm to vary the rigidity about the driving shaft member.

According to the above configuration, an expansion/contraction amount of the expansion/contraction spring is varied relative to a swinging angle of the first swinging arm. In this manner, a structure that varies rigidity about the driving shaft member may be realized.

In the above configuration, the rigidity variable portion may be constituted by a driven shaft member that is arranged parallel to the driving shaft member, an interlocking swinging member that is swingably supported about the driven shaft member and connected to the first swinging arm via a power transmission portion to swing in an interlocking manner with swinging of the first swinging arm while swinging at an interlocking swinging angle smaller than a swinging angle of the first swinging arm, a rigidity adjustment shaft member that is arranged at a position near a periphery of the interlocking swinging member and arranged parallel to the driven shaft member, a rigidity adjustment shaft pivoting portion that pivots the rigidity adjustment shaft member, a pivoting member that is connected to the rigidity adjustment shaft member to pivot with the rigidity adjustment shaft member, and the expansion/contraction spring, a portion corresponding to a first end of the expansion/contraction spring may be connected to a spring fixing end of the pivoting member that is at a position away from the rigidity adjustment shaft member, a portion corresponding to a second end of the expansion/contraction spring may be connected to a spring swinging end that is at a position near the periphery of the interlocking swinging member, the spring swinging end being coaxial with the rigidity adjustment shaft member at the position when the interlocking swinging angle is zero, the expansion/contraction spring connected to the spring fixing end and the spring swinging end may have a free length when the interlocking swinging angle is zero, and the control portion may control the rigidity adjustment shaft pivoting portion to adjust a rigidity adjustment angle according to the interlocking swinging angle to adjust the apparent spring constant of the expansion/contraction spring seen from the interlocking swinging member, the rigidity adjustment angle being an angle formed between a virtual tangential line and a virtual line, the virtual tangential line representing a tangential line that is set on a circumference of a virtual interlocking swinging circle serving as a circle having a distance between the driven shaft member and the rigidity adjustment shaft member as a radius about the driven shaft member and that is set at a position of the rigidity adjustment shaft member, the virtual line connecting the spring swinging end and the spring fixing end to each other when the interlocking swinging angle is zero.

According to the above configuration, the rigidity variable portion including the expansion/contraction spring may be specifically realized. In addition, since an apparent spring constant may be adjusted only by controlling the rigidity adjustment shaft pivoting portion with the control portion and pivoting the pivoting member, the apparent spring constant may be easily adjusted.

In the above configuration, the control portion may adjust the rigidity adjustment angle such that a resonance point of the expansion/contraction spring coincides with a swinging frequency of a swinging object including the first swinging arm, based on a swinging frequency of the first swinging arm about the driving shaft member, inertia moment about the driving shaft member in the swinging object, a spring constant of the expansion/contraction spring, the free length of the expansion/contraction spring, a distance between the driven shaft member and the rigidity adjustment shaft member, and the interlocking swinging angle.

According to the above configuration, a rigidity adjustment angle (a pivoting angle of the pivoting member) may be automatically adjusted using the control portion to an appropriate angle corresponding to a swinging object including the first swinging arm. Accordingly, a generated torque may be automatically adjusted when the rigidity of a joint that performs swinging motion is automatically adjusted. In addition, even when the first swinging arm is caused to perform swinging motion by an electric motor, the swinging motion may be assisted at an appropriate torque. Therefore, the consumption power of the electric motor for swinging may be further reduced. Moreover, even when the swinging arm is not caused to swing by the electric motor but is caused to swing by a user himself/herself, the swinging motion may be assisted at an appropriate torque. Therefore, a user's load may be further reduced.

In the above aspect, the walking-ability assisting device may further include: a first driving portion that swings the first swinging arm about the driving shaft member based on a control signal from the control portion.

According to the above configuration, since the first driving portion swings the first swinging arm, a load may be further reduced when a user walks or runs.

In the above aspect, the walking-ability assisting device may further include: a second swinging arm that is swingably supported about the driving shaft member; a second angle detection portion that detects a second swinging angle as a swinging angle of the second swinging arm; a second driving portion that swings the second swinging arm about the driving shaft member based on a control signal from the control portion; and a swinging link member that is connected to the first swinging arm and the second swinging arm to operate based on the first swinging angle of the first swinging arm and the second swinging angle of the second swinging arm.

According to the above configuration, since the first swinging arm may assist the motion of the femoral part of a user and the second swinging arm may assist the crus part of the user, a load may be further reduced when the user walks or runs.

According to a third aspect of the invention, there is provided a method for controlling rigidity of a swinging joint, the swinging joint including a driving shaft member, a first swinging arm that is swingably supported about the driving shaft member, a driven shaft member that is arranged parallel to the driving shaft member, an interlocking swinging member that is connected to the first swinging arm via a power transmission portion to swing about the driven shaft member in an interlocking manner with swinging of the first swinging arm while swinging at an interlocking swinging angle smaller than a swinging angle of the first swinging arm, an elastic body that is connected to the interlocking swinging member to generate an urging force corresponding to the interlocking swinging angle, the urging force being generated in a direction opposite to an interlocking swinging direction of the interlocking swinging member, a rigidity variable portion that varies rigidity of the elastic body seen from the interlocking swinging member, and a control portion that controls the rigidity variable portion, the method including: adjusting the rigidity of the elastic body seen from the interlocking swinging member according to the interlocking swinging angle using the control portion and the rigidity variable portion.

According to the above aspect, an apparent spring constant variable portion is controlled according to an interlocking swinging angle using the control portion. Therefore, since a degree of a torque required for assisting swinging motion is automatically adjusted for the swinging motion of a swinging object including a swinging arm, the torque may be adjusted without any trouble. In addition, since a torque required for assisting swinging motion is generated using an expansion/contraction spring, consumption power or a user's load may be further reduced.

In addition, in the above aspect, the elastic body may be an expansion/contraction spring, and the rigidity variable portion may be an apparent spring constant variable portion that varies an apparent spring constant of the expansion/contraction spring seen from the interlocking swinging member.

According to the above configuration, since the use of the expansion/contraction spring as the elastic body makes it possible to secure an optimum energy reservation amount and easily adjust a spring constant (rigidity) for a user's action such as walking and running, energy may be smoothly reserved and output.

In the above configuration, the apparent spring constant variable portion may be constituted by a rigidity adjustment shaft member that is arranged at a position near a periphery of the interlocking swinging member and arranged parallel to the driven shaft member, a rigidity adjustment shaft pivoting portion that pivots the rigidity adjustment shaft member, a pivoting member that is connected to the rigidity adjustment shaft member to pivot with the rigidity adjustment shaft member, and the expansion/contraction spring, a portion corresponding to a first end of the expansion/contraction spring may be connected to a spring fixing end of the pivoting member that is at a position away from the rigidity adjustment shaft member, a portion corresponding to a second end of the expansion/contraction spring may be connected to a spring swinging end that is at a position near the periphery of the interlocking swinging member, the spring swinging end being coaxial with the rigidity adjustment shaft member at the position when the interlocking swinging angle is zero, the expansion/contraction spring connected to the spring fixing end and the spring swinging end may have a free length when the interlocking swinging angle is zero, and the rigidity adjustment shaft pivoting portion may be controlled using the control portion to adjust a rigidity adjustment angle according to the interlocking swinging angle to adjust the apparent spring constant of the expansion/contraction spring seen from the interlocking swinging member, the rigidity adjustment angle being an angle formed between a virtual tangential line and a virtual line, the virtual tangential line representing a tangential line that is set on a circumference of a virtual interlocking swinging circle serving as a circle having a distance between the driven shaft member and the rigidity adjustment shaft member as a radius about the driven shaft member and that is set at a position of the rigidity adjustment shaft member, the virtual line connecting the spring swinging end and the spring fixing end to each other when the interlocking swinging angle is zero.

According to the above configuration, the apparent spring constant variable portion including the expansion/contraction spring may be specifically realized. In addition, since an apparent spring constant may be adjusted only by controlling the rigidity adjustment shaft portion with the control portion and pivoting the pivoting member, the apparent spring constant may be easily adjusted.

In the above configuration, the rigidity adjustment angle may be adjusted using the control portion such that a resonance point of the expansion/contraction spring coincides with a swinging frequency of a swinging object including the first swinging arm, based on a swinging frequency of the first swinging arm about the driving shaft member, inertia moment about the driving shaft member in the swinging object, a spring constant of the expansion/contraction spring, the free length of the expansion/contraction spring, a distance between the driven shaft member and the rigidity adjustment shaft member, and the interlocking swinging angle.

According to the above configuration, a rigidity adjustment angle (a pivoting angle of the pivoting member) may be automatically adjusted using the control portion to an appropriate angle corresponding to a swinging object including the swinging arm. Accordingly, a generated torque may be automatically adjusted when the rigidity of a joint that performs swinging motion is automatically adjusted. In addition, even when the swinging arm is caused to perform swinging motion by an electric motor, the swinging motion may be assisted at an appropriate torque. Therefore, the consumption power of the electric motor for swinging may be further reduced. Moreover, even when the swinging arm is not caused to swing by the electric motor but is caused to swing by a user himself/herself, the swinging motion may be assisted at an appropriate torque. Therefore, a user's load may be further reduced.

### BRIEF DESCRIPTION OF THE DRAWINGS

Features, advantages, and technical and industrial significance of exemplary embodiments of the invention will be described below with reference to the accompanying drawings, in which like numerals denote like elements, and wherein:

FIG. 1 is an exploded perspective view describing the schematic shapes and the assembling positions of the respective constituents of the swinging joint device of a first embodiment;

FIG. 2 is a perspective view of the swinging joint device in which the respective constituents shown in FIG. 1 are assembled together;

FIG. 3 is a view describing a state in which a user (whose arms are not shown) wears the swinging joint device shown in FIG. 2;

FIG. 4 is a view describing a swinging state of a femoral swinging arm and a swinging example of a crus swinging arm;

FIG. 5 is a cross-sectional view taken along line V-V in FIG. 4 and describing the configuration of a spring unit;

FIG. 6 is a view describing the operation of the spring unit when a force is applied to the spring unit in a contraction direction;

FIG. 7 is a view describing the operation of the spring unit when a force is applied to the spring unit in an expansion direction;

FIG. 8 is a perspective view showing the periphery of the spring unit when a swinging angle of the femoral swinging arm is zero;

FIG. 9 is a perspective view showing the periphery of the spring unit when the femoral swinging arm swings forward from a state shown in FIG. 8;

FIG. 10 is a view describing a state in which a schematically-shown expansion/contraction spring expands/contracts according to the swinging of an interlocking swinging member when a driven shaft member, a rigidity adjustment shaft member, and a spring fixing end are aligned;

FIG. 11 is a view describing a state in which, in contrast to FIG. 10, the schematically-shown expansion/contraction spring expands/contracts according to the swinging of the interlocking swinging member when a pivoting angle of the spring unit is changed;

FIG. 12 is a view describing the input/output of a control portion;

FIG. 13 is a flowchart describing an example of the processing procedure of the control portion;

FIG. 14 is a view describing a procedure for calculating a rigidity adjustment angle for adjusting an apparent spring constant; and

FIG. 15 is a view describing an example in which the interlocking swinging member has two spring units.

### DETAILED DESCRIPTION OF EMBODIMENTS

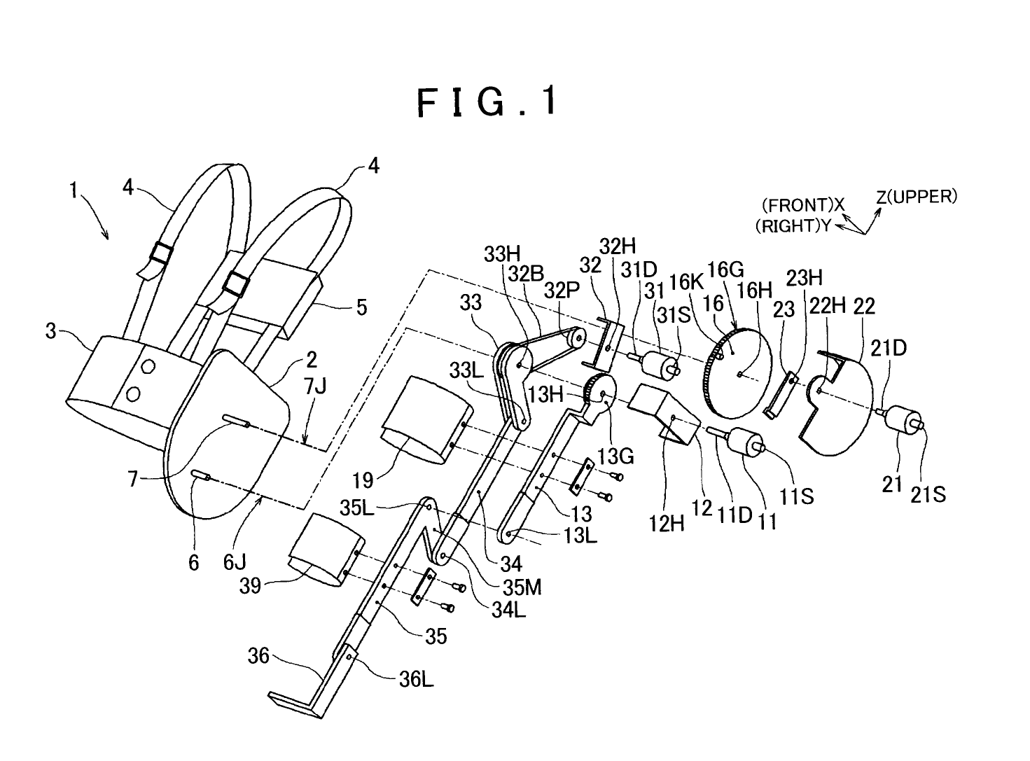

Hereinafter, a description will be given, with reference to the drawings, of a first embodiment as an embodiment for carrying out the invention. Note that when respective figures describe X, Y, and Z axes, the X, Y, and Z axes are orthogonal to each other, a Z-axis direction indicates a vertically-upward direction, an X-axis direction indicates a front direction relative to a user (user wearing a swinging joint device), and a Y-axis direction indicates a right direction relative to the user. Note that in the specification, a “femoral swinging arm 13” and a “crus swinging arm 33” shown in FIG. 1 exemplify a “first swinging arm” and a “second swinging arm,” respectively. In addition, a “rotation angle detection portion 11S” and a “rotation angle detection portion 31S” exemplify a “first angle detection portion” and a “second angle detection portion,” respectively. Moreover, an “electric motor 11” and an “electric motor 31” exemplify a “first driving portion” and a “second driving portion,” respectively. Further, a “base portion 2” exemplifies a “waist-side attachment portion.” Furthermore, a “swinging joint device 1” exemplifies a “walking-ability assisting device.” Furthermore, an example in which a driving shaft member 6 is a protruding member is shown in an explanation below. However the driving shaft member 6 may be a shaft having a protruding shape or be a hollow (a hole) supporting a shaft. Therefore, a description of “supported about the driving shaft member 6” means the same as “supported about a driving axis 6J as a central axis of the driving shaft member 6”. Furthermore, a “crus relaying arm 34” and a “crus arm 35” exemplify a “swinging link member”. Furthermore, an “elastic body” includes a “expansion/contraction spring 23K”, and the expansion/contraction spring is used in the explanation below.

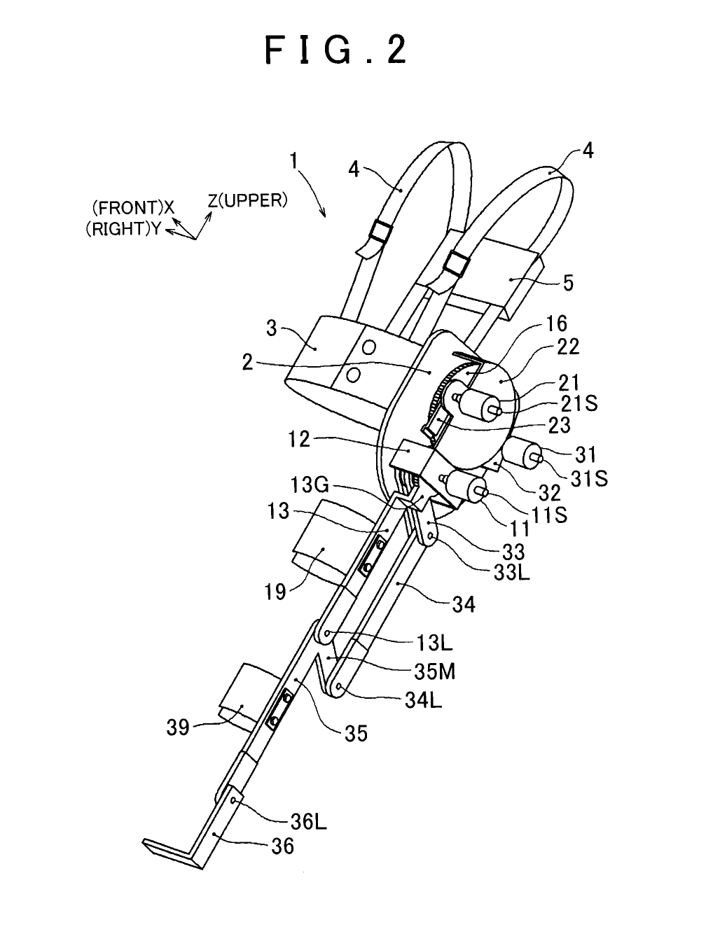

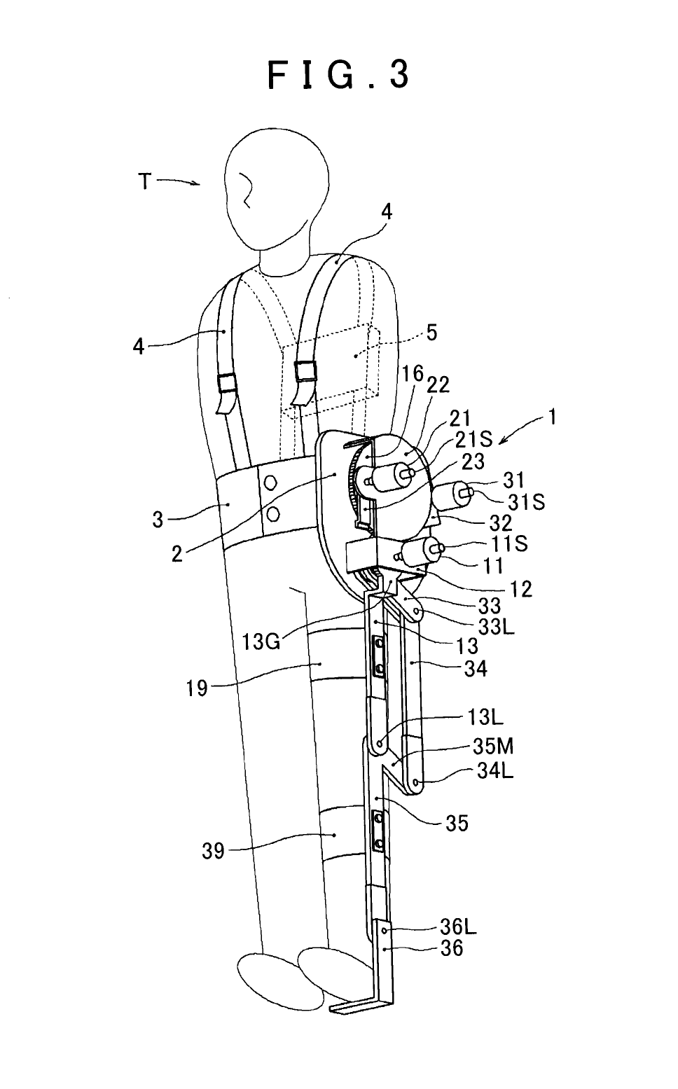

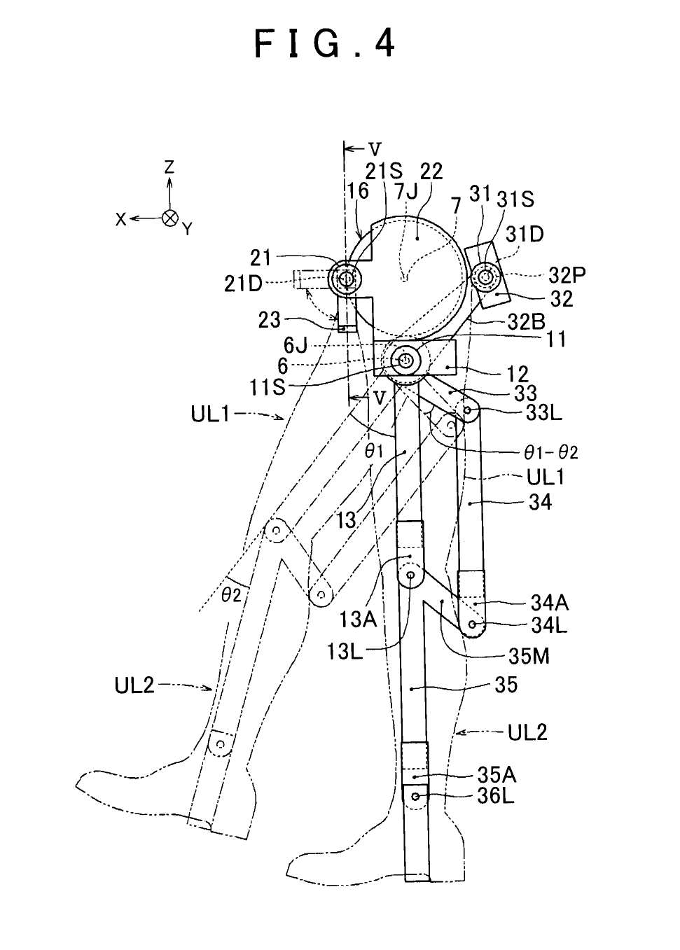

The swinging joint device 1 of the first embodiment is attached to one leg (the left leg in the first embodiment) of a user to assist a user's action such as walking and running. As shown in FIG. 1, the swinging joint device 1 is constituted by a user attachment portion indicated by symbols 2, 3, 4, 5, 6, and 7, a femoral swinging portion indicated by symbols 11, 12, 13, and 19, a rigidity adjustment portion indicated by symbols 16, 21, 22, and 23, and a crus swinging portion indicated by symbols 31, 32, 32P, 32B, 33, 34, 35, 36, and 39. Note that FIG. 1 is an exploded perspective view showing the shapes, the assembling positions, or the like of the respective constituents of the swinging joint device 1, and FIG. 2 shows the swinging joint device 1 in a state in which the respective constituents are assembled together. In addition, FIG. 3 shows a state in which a user wears the swinging joint device 1, and FIG. 4 shows a swinging example of the femoral swinging arm 13 and the crus swinging arm 33.

The base portion 2 is fixed to a waist attachment portion 3 and is a member serving as a base (board) for holding the femoral swinging portion, the rigidity adjustment portion, and the crus swinging portion. In addition, the base portion 2 has the driving shaft member 6 extending nearly parallel to the Y axis at a position corresponding to the lateral side of the hip joint of a user wearing the swinging joint device 1 and has a driven shaft member 7 arranged parallel to the driving shaft member 6 on the upper side of the driving shaft member 6. Note that the driving shaft member 6 is inserted into a through-hole 33H of a crus swinging arm 33 that will be described later, and then inserted into a through-hole 13H of a femoral swinging arm 13. In addition, the driven shaft member 7 is inserted into a through-hole 16H of an interlocking swinging member 16. Note that the driving axis 6J indicates the central axis of the driving shaft member 6 and a driven axis 7J indicates the central axis of the driven shaft member 7.

The waist attachment portion 3 is a member wound on and fixed to the waist of a user and configured to be adjustable according to a size of the waist of the user. In addition, the waist attachment portion 3 is fixed to the base portion 2 and connected to one and the other ends of shoulder belts 4.

The shoulder belts 4 are connected to the front-surface side and the back-surface side of the waist attachment portion 3 at their ends (one and other ends), configured to be capable of adjusting their lengths, and attached to the control unit 5. A user may carry the control unit 5 on his/her back like a backpack by adjusting lengths of the shoulder belts 4 and putting the control unit 5 on the back.

The control unit 5 accommodates a control portion that controls the electric motors 11, 21, and 31, a battery that supplies power to the control portion and the electric motors 11, 21, and 31, or the like.

The femoral swinging arm 13 (exemplifying the first swinging arm) is constituted by a disc portion 13G having gear teeth at its peripheral surface and an arm portion extending downward from the disc portion 13G. The disc portion 13G has the through-hole 13H at its center, and the driving shaft member 6 is inserted into the through-hole 13H. Accordingly, the femoral swinging arm 13 is swingably supported about the driving shaft member 6. In addition, the through-hole 13H of the femoral swinging arm 13 is arranged at a position corresponding to the lateral side of the hip joint of a user, and a link hole 13L provided at the lower end of the femoral swinging arm 13 is arranged at a position corresponding to the lateral side of the knee joint of the user. Note that a downwardly-extending length of the femoral swinging arm 13 is configured to be adjustable, and a user is capable of adjusting a vertical position of the link hole 13L according to a position of his/her knee joint. In addition, the femoral swinging arm 13 is attached to a femoral attachment portion 19. The femoral attachment portion 19 is put on the femoral part (the circumference of the thigh) of a user to facilitate the attachment of the femoral swinging arm 13 to the femoral part of the user.

A bracket 12 is a member for fixing the electric motor 11 such that the rotation shaft of the electric motor 11 is coaxial with the driving shaft member 6, and is fixed to the base portion 2 such that the through-hole 12H is coaxial with the driving shaft member 6. Note that the bracket 12 is fixed to the base portion 2 after the through-hole 33H of the crus swinging arm 33 is first fitted in the driving shaft member 6 and then the through-hole 13H of the femoral swinging arm 13 is fitted in the driving shaft member 6.

The electric motor 11 has a speed reducer 11D at its tip end, and the speed reducer 11D is inserted into the through-hole 12H of the bracket 12 to be attached at the center of the disc portion 13G of the femoral swinging arm 13. In addition, the electric motor 11 is fixed to the bracket 12. Moreover, the electric motor 11 receives power from the battery and the control portion accommodated in the control unit 5 together with driving signals. Then, the electric motor 11 may swing the femoral swinging arm 13 back and forth about the driving shaft member 6 relative to the bracket 12 (i.e., the base portion 2) (see FIG. 4). Further, the electric motor 11 has the rotation angle detection portion 11S such as an encoder. The rotation angle detection portion 11S outputs a signal corresponding to a rotation angle of the shaft of the electric motor 11 to the control portion. The control portion is capable of detecting a rotation angle of the speed reducer 11D based on a detection signal from the rotation angle detection portion 11S and a speed reduction ratio of the speed reducer 11D and capable of detecting a swinging angle of the femoral swinging arm 13. Note that the bracket 12 may have an angle detection portion (angular sensor) that detects a swinging angle of the femoral swinging arm 13 relative to the bracket 12 or may have an angle detection portion (angular sensor) that detects a swinging angle of the crus swinging arm 33 relative to the bracket 12. In addition, the bracket 12 may have an angle detection portion that detects a swinging angle of the interlocking swinging member 16 instead of the angle detection portion that detects a swinging angle of the femoral swinging arm 13.

The crus swinging arm 33 has the through-hole 33H into which the driving shaft member 6 is inserted. When the driving shaft member 6 is inserted into the through-hole 33H, the crus swinging arm 33 is swingably supported about the driving shaft member 6. A belt 32B is put on the crus swinging arm 33, and power is transmitted from a power transmission portion constituted by the electric motor 31, a pulley 32P, and a belt 32B to swing the crus swinging arm 33 about the driving shaft member 6.

The crus relaying arm 34 has an upper end swingably connected to the tip end of the crus swinging arm 33 and a lower end swingably connected to the end of a parallel link forming portion 35M on the upper-end side of the crus arm 35. Note that a downwardly-extending length of the crus relaying arm 34 is configured to be adjustable. That is, a length of the crus relaying arm 34 is adjusted according to an adjusted length of the femoral swinging arm 13.

The crus arm 35 is formed into a substantially reverse L-shape and has a link hole 35L, which is connected to the link hole 13L at the lower end of the femoral swinging arm 13, at a position corresponding to an L-shaped bending portion. Accordingly, the crus arm 35 is formed such that one end of the parallel link forming portion 35M on an upper-end side is swingably connected to the lower end of the crus relaying arm 34 and the other end of the parallel link forming portion 35M is swingably connected to the lower end of the femoral swinging arm 13. In addition, the crus arm 35 has a lower end to which to the upper end of a foot holding portion 36 is swingably connected. Note that a downwardly-extending length of the crus arm 35 is configured to be adjustable so as to match the crus of a user. In addition, the foot holding portion 36 is formed into a substantially L-shape and has a lower end positioned at the bottom of the foot of a user. Moreover, the crus arm 35 is attached to a crus attachment portion 39. The crus attachment portion 39 is put on the crus (the circumference of the calf) of a user to facilitate the attachment of the crus arm 35 to the crus part of the user.

A bracket 32 is a member for fixing the electric motor 31 and fixed to the base portion 2. In addition, the bracket 32 has a through-hole 32H.

The electric motor 31 has a speed reducer 31D at its tip end, and the speed reducer 31D is inserted into the through-hole 32H of the bracket 32. In addition, the speed reducer 31D is attached to the pulley 32P, and the belt 32B is put between the pulley 32P and the crus swinging arm 33. Moreover, the electric motor 31 receives power from the battery and the control portion accommodated in the control unit 5 together with driving signals. Then, the electric motor 31 may swing the crus swinging arm 33 back and forth about the driving shaft member 6 via the pulley 32P and the belt 32B (see FIG. 4). Further, the electric motor 31 has the rotation angle detection portion 31S such as an encoder. The rotation angle detection portion 31S outputs a signal corresponding to a rotation angle of the shaft of the electric motor 31 to the control portion. The control portion is capable of detecting a rotation angle of the crus swinging arm 33 based on a detection signal from the rotation angle detection portion 31S, a speed reduction ratio of the speed reducer 31D, and a pulley ratio and capable of detecting a swinging angle of the crus swinging arm 33.

Next, a description will be given, with reference to FIG. 4, of the operation of assisting the swinging of a femoral part UL1 of a user wearing the femoral swinging arm 13 and the operation of assisting the swinging of a crus part UL2 of the user wearing the crus arm 35. The femoral swinging arm 13 swings about the driving shaft member 6 when receiving power from the electric motor 11. Similarly, the crus swinging arm 33 swings about the driving shaft member 6 when receiving power from the electric motor 31. In addition, the femoral swinging arm 13, the crus swinging arm 33, the crus relaying arm 34, and the parallel link forming portion 35M (of the crus arm 35) constitute a parallel link formed into a parallelogram. Accordingly, the crus relaying arm 34 and the crus arm 35 correspond to swinging link members that are connected to the femoral swinging arm 13 and the crus swinging arm 33 and operates based on a swinging angle (angle θ1 in FIG. 4) of the femoral swinging arm 13 and a swinging angle (angle θ1-θ2 in FIG. 4) of the crus swinging arm 33. Note that the positions of the femoral swinging arm 13, the crus swinging arm 33, the crus relaying arm 34, and the crus arm 35 indicated by solid lines in FIG. 4 are set as the initial positions (positions where a user is at a standstill in an upright posture) of the respective arms.

When the femoral swinging arm 13 swings forward at the angle θ1 from its initial position, the femoral part UL1 of a user may swing forward at the angle θ1 as shown in FIG. 4. At the same time, when the crus swinging arm 33 swings forward at the angle (θ1-θ2) from its initial position, the crus part UL2 of the user may swing forward so as to tilt at the angle θ2 relative to the femoral swinging arm 13 as shown in FIG. 4. Since the swinging motion of the femoral swinging arm 13 with the electric motor 11 and the swinging motion of the crus swinging arm 33 with the electric motor 31 may be separately controlled, the user is allowed to freely adjust the angle θ1 and the angle θ2 as he/she wants. In addition, since the swinging of the femoral part that requires a large torque may be based on both torques of the electric motors 11 and 31 according to the configuration, a large motor is not required.

In addition, when the femoral swinging arm 13 swings, the interlocking swinging member 16 swings (pivots back and forth) (i.e., swinging motion) in an interlocking manner. Then, the energy of the swinging motion is accumulated in a spring unit 23 via the interlocking swinging member 16 and used to perform swinging motion in an opposite direction. That is, energy generated when the femoral swinging arm 13 swings forward is accumulated in the spring unit 23 and used when the femoral swinging arm 13 swings backward, and energy generated when the femoral swinging arm 13 swings backward is accumulated in the spring unit and used when the femoral swinging arm 13 swings forward. Next, a description will be given of the rigidity adjustment portion including the spring unit 23.

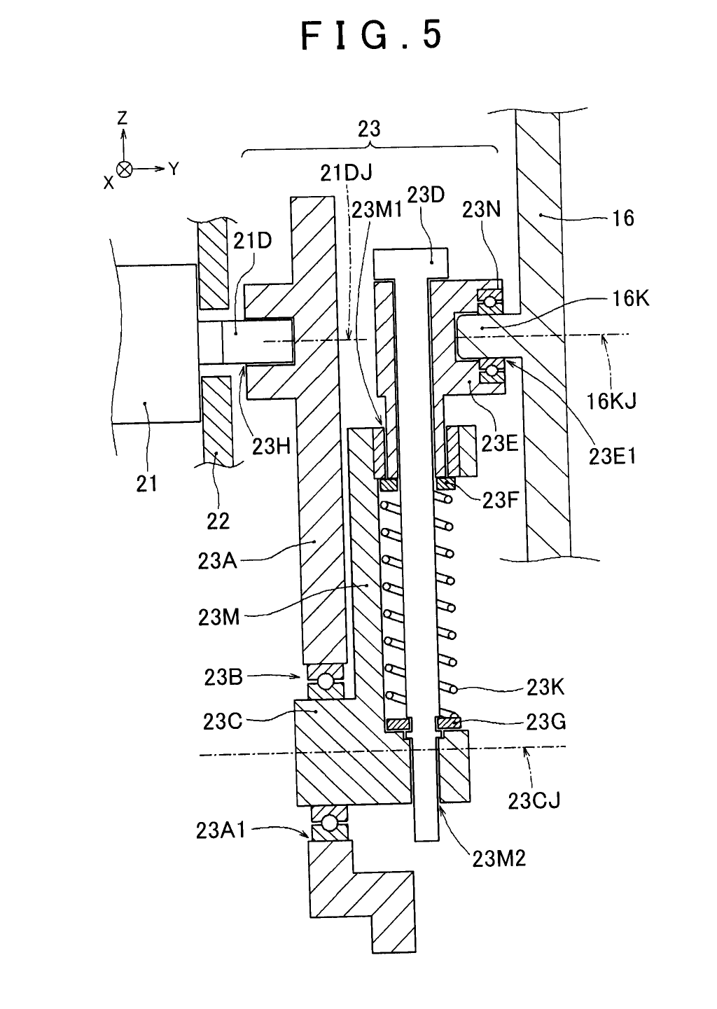

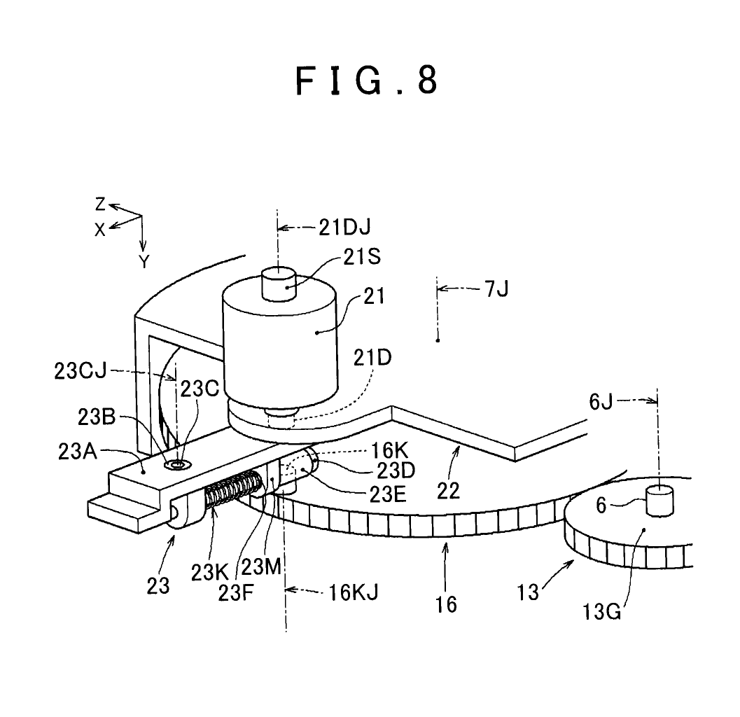

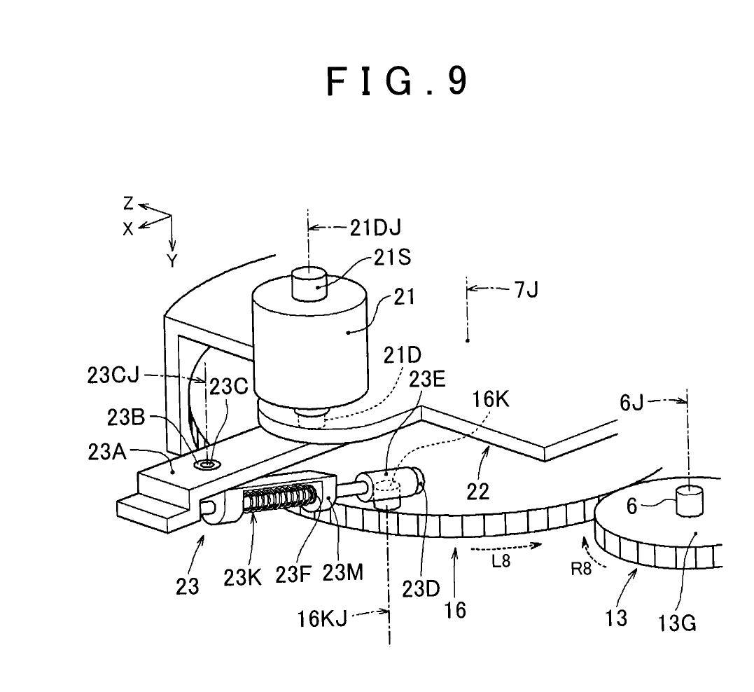

A bracket 22 is a member that fixes the electric motor 21 at a position (see FIGS. 8 and 9) at which a rigidity adjustment shaft member 21D (in this case, the speed reducer) of the electric motor 21 is coaxial with a spring engagement member 16K provided on the periphery of the interlocking swinging member 16 when an interlocking swinging angle is zero, and is fixed to the base portion 2. In addition, the bracket 22 has a through-hole 2214 at the position at which the rigidity adjustment shaft member 21D is coaxial with the spring engagement member 16K of the interlocking swinging member 16 when the interlocking swinging angle is zero. Note that a rigidity adjustment axis 21DJ (see FIGS. 5 to 7) serving as the rotation axis of the rigidity adjustment shaft member 21D is parallel to the driving axis 6J and the driven axis 7J.

The interlocking swinging member 16 is a disc-shaped member having gear teeth 16G at its peripheral surface. The interlocking swinging member 16 has the through-hole 16H at its center, and the driven shaft member 7 is inserted into the through-hole 16H. Accordingly, the interlocking swinging member 16 is swingably supported about the driven shaft member 7. In addition, the gear teeth at the peripheral surface of the disc portion 13G of the femoral swinging arm 13 and the gear teeth at the peripheral surface of the interlocking swinging member 16 mesh with each other, and the interlocking swinging member 16 swings with the swinging motion of the femoral swinging arm 13. Moreover, the diameter of the interlocking swinging member 16 is set to be substantially larger than that of the disc portion 13G, and the ratio of the gear teeth of the disc portion 13G to the gear teeth of the interlocking swinging member 16 is set at, for example, 1:10. In this case, for example, when the femoral swinging arm 13 swings at a swinging angle of 60°, the interlocking swinging member 16 swings at a swinging angle of 6° in an interlocking manner. Moreover, the spring engagement member 16K (exemplifying a spring swinging end, see FIG. 1) is provided at a position near the periphery of the interlocking swinging member 16, i.e., at the position (see FIGS. 8 and 9) at which the spring engagement member 16K is coaxial with the rigidity adjustment shaft member 21D (the speed reducer provided on the shaft of the electric motor 21) when the interlocking swinging angle is zero. The spring engagement member 16K is connected to one end of the expansion/contraction spring of a spring unit 23.

The electric motor 21 has the rigidity adjustment shaft member 21D at its tip end, and the rigidity adjustment shaft member 21D is inserted into the through-hole 22H of the bracket 22 to be attached to an attachment portion 23H of the spring unit 23. In addition, the electric motor 21 is fixed to the bracket 22. Moreover, the electric motor 21 receives power together with driving signals from the battery and the control portion accommodated in the control unit 5. Further, the electric motor 21 may pivot the spring unit 23 about the rigidity adjustment shaft member 21D relative to the bracket 22 (i.e., the base portion 2) (see FIG. 4). Furthermore, the electric motor 21 has the rotation angle detection portion 21S such as an encoder. The rotation angle detection portion 21S outputs a signal corresponding to a rotation angle of the shaft of the electric motor 21 to the control portion. Meanwhile, the control portion is capable of detecting a rotation angle of the rigidity adjustment shaft member 21D based on a detection signal from the rotation angle detection portion 21S and a speed reduction ratio of the rigidity adjustment shaft member 21D and capable of detecting a pivoting angle of the spring unit 23 (a pivoting member 23A). Note that the bracket 22 may have an angle detection portion (angular sensor) that detects a pivoting angle of the spring unit 23 (the pivoting member 23A) relative to the bracket 22. Note that a description will be given in detail of the spring unit 23 below.

As shown in FIG. 5 (a cross-sectional view taken along line V-V in FIG. 4), the spring unit 23 is constituted by a pivoting member 23A having the attachment portion 23H, a bearing 23B, a swinging following member 23M having a swinging following shaft member 23C, a shaft 23D, an expansion/contraction transmission member 23E, washers 23F and 23G, and the expansion/contraction spring 23K.

The pivoting member 23A allows a rigidity adjustment shaft member 21D of the electric motor 21 to be fitted in the attachment portion 23H provided near its one end and pivots about a rigidity adjustment axis 21DJ. In addition, the pivoting member 23A has a through-hole 23A1, which is used to receive the bearing 23B and the swinging following shaft member 23C, at the other end (at a position away from the rigidity adjustment shaft member) of the pivoting member 23A.

The swinging following shaft member 23C (exemplifying a spring fixing end) is attached via the bearing 23B at a position away from the rigidity adjustment shaft member 21D of the pivoting member 23A. Accordingly, the swinging following member 23M having the swinging following shaft member 23C is supported so as to be capable of pivoting about a spring support axis 23CJ parallel to the rigidity adjustment axis 21DJ. In addition, the swinging following member 23M has through-holes 23M1 and 23M2, which are used to receive the shaft 23D, in a direction orthogonal to the rigidity adjustment axis 21DJ.

The expansion/contraction transmission member 23E, the washer 23F, the expansion/contraction spring 23K, and the washer 23G are fitted to the shaft 23D, and the shaft 23D is inserted into the through-holes 23M1 and 23M2 of the swinging following member 23M. An attachment portion 23E1 of the expansion/contraction transmission member 23E receives, via a bearing 23N, the spring engagement member 16K (see FIG. 1) provided on the periphery of the interlocking swinging member 16. Note that when the rigidity adjustment axis 21DJ is coaxial with a spring swinging axis 16KJ serving as the central axis of the spring engagement member 16K, the expansion/contraction spring 23K has a free length and is in a state of being neither contracted nor expanded.

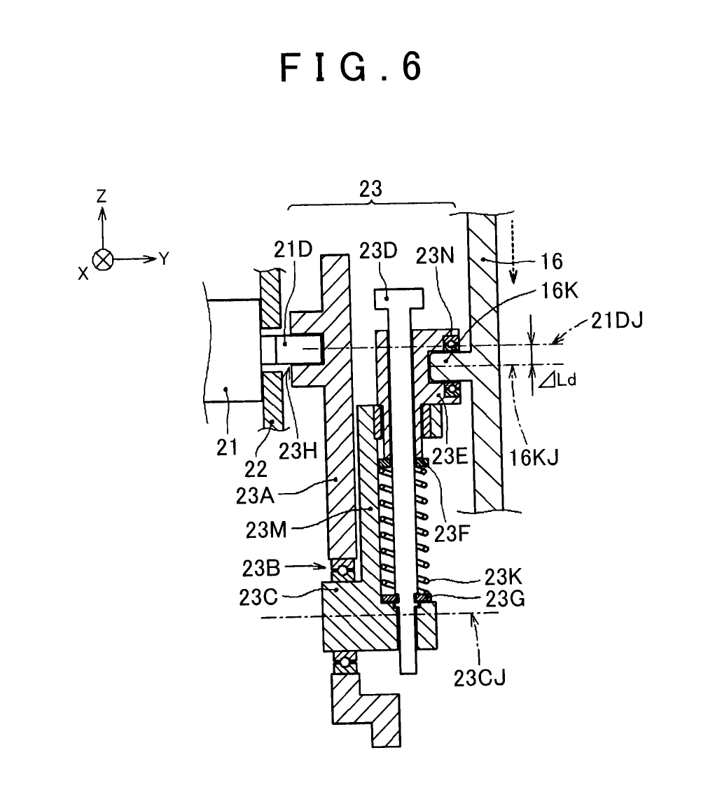

When the interlocking swinging member 16 moves downward from a state shown in FIG. 5 with the above configuration, the spring engagement member 16K of the spring unit 23 pushes the expansion/contraction transmission member 23E and the washer 23F downward as shown in FIG. 6. Then, the expansion/contraction spring 23K is contracted, and an urging force of the expansion/contraction spring 23K is applied in a direction in which a distance ΔLd between the rigidity adjustment axis 21DJ and the spring swinging axis 16KJ becomes zero.

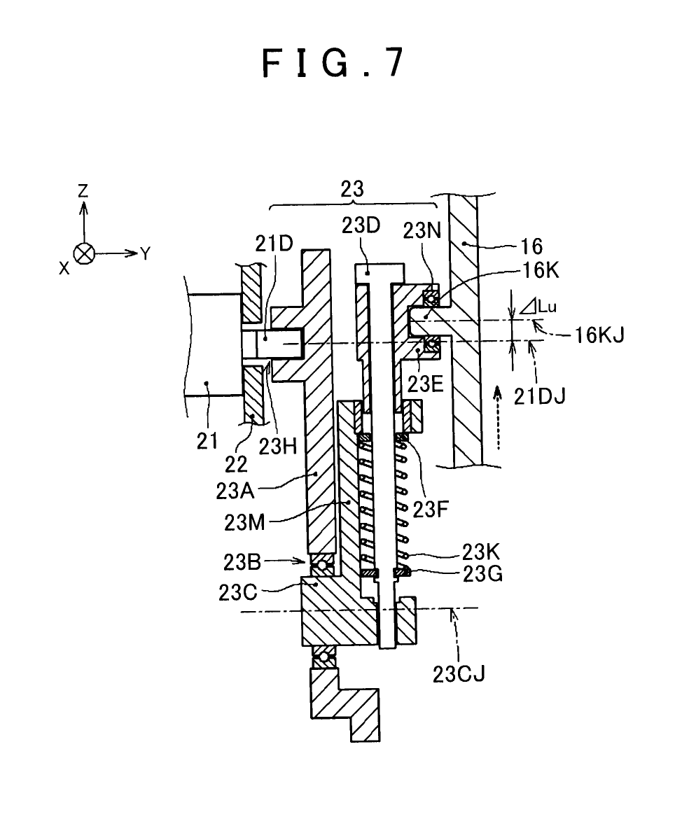

On the other hand, when the interlocking swinging member 16 moves upward from the state shown in FIG. 5, the spring engagement member 16K pushes the expansion/contraction transmission member 23E, the shaft 23D, and the washer 23G upward as shown in FIG. 7. Then, the expansion/contraction spring 23K is contracted, and an urging force of the expansion/contraction spring 23K is applied in a direction in which a distance ΔLu between the rigidity adjustment axis 21DJ and the spring swinging axis 16KJ becomes zero.

FIG. 8 is a perspective view of the periphery of the spring unit 23 when the swinging angle of the femoral swinging arm 13 is zero. When the swinging angle of the femoral swinging arm 13 is zero, the rigidity adjustment axis 21DJ is coaxial with the spring swinging axis 16KJ and the expansion/contraction spring 23K has a free length. The pivoting member 23A of the spring unit 23 is capable of pivoting about the rigidity adjustment axis 21DJ of the rigidity adjustment shaft member 21D of the electric motor 21, and a pivoting angle of the pivoting member 23A is adjusted by the electric motor 21. In addition, the swinging following member 23M of the spring unit 23 is capable of pivoting about the spring support axis 23CJ.

FIG. 9 shows a case in which the femoral swinging arm 13 swings in a direction indicated by symbol R8 from a state shown in FIG. 8 and shows a case in which the interlocking swinging member 16 swings in a direction indicated by symbol L8 in an interlocking manner. When the femoral swinging arm 13 swings in the direction indicated by symbol R8, the interlocking swinging member 16 meshing with the gear teeth on the periphery of the disc portion 13G of the femoral swinging arm 13 swings in the direction indicated by symbol L8 in an interlocking manner. Then, the spring engagement member 16K on the periphery of the interlocking swinging member 16 moves in a direction away from the rigidity adjustment axis 21DJ and pulls the expansion/contraction transmission member 23E in a direction away from the spring support axis 23CJ. Then, the expansion/contraction spring 23K is contracted (see FIG. 7), and an urging force generated in the expansion/contraction spring 23K acts as a force (a force for pivoting the interlocking swinging member 16 in a direction opposite to the direction indicated by symbol L8) for pivoting the interlocking swinging member 16 in a direction in which the spring swinging axis 16KJ is coaxial with the rigidity adjustment axis 21DJ.

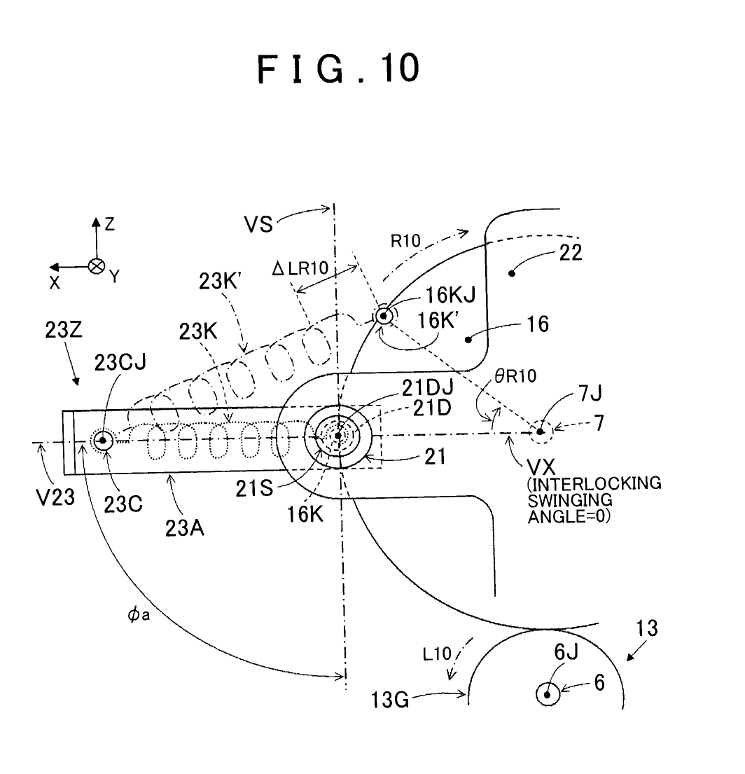

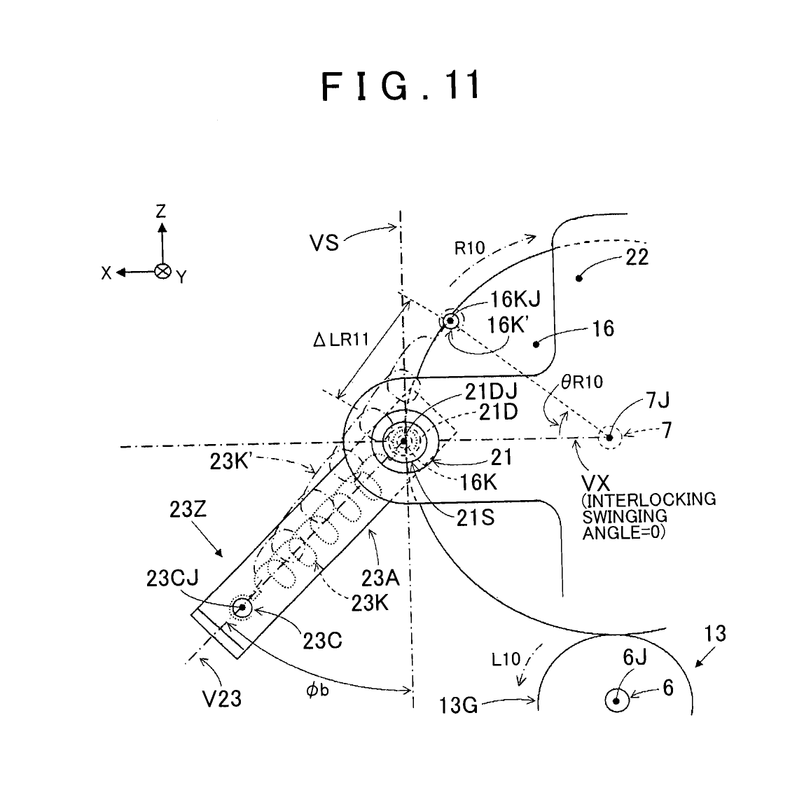

Next, a description will be given of rigidity adjustment angles with reference to FIGS. 10 and 11. Note that each of FIGS. 10, 11, and 14 shows a schematic diagram of a spring unit 23Z in which the structure of the spring unit 23 shown in FIG. 5 is simplified. In each of the schematic diagrams of the spring unit 23Z, only the pivoting member 23A, the swinging following shaft member 23C, and the expansion/contraction spring 23K in the configuration of the spring unit 23 shown in FIG. 5 are left. The one end of the expansion/contraction spring 23K engages with the swinging following shaft member 23C, and the other end thereof engages with the spring engagement member 16K. In addition, when the interlocking swinging angle shown in FIG. 10 is zero and the rigidity adjustment axis 21DJ is coaxial with the spring swinging axis 16KJ, the expansion/contraction spring 23K has a free length at which the expansion/contraction spring 23K is neither expanded nor contracted.

In FIGS. 10 and 11, a tangential line that is set on the periphery of a virtual interlocking swinging circle (in the embodiment, the peripheral circle of the interlocking swinging member) serving as a circle having a distance between the driven shaft member 7 and the rigidity adjustment shaft member 21D as a radius about the driven shaft member 7, and that is set at the position of the rigidity adjustment shaft member 21D is indicated as a virtual tangential line VS. In addition, the line that connects the spring fixing end (the swinging following shaft member 23C as an example) and the spring swinging end (the spring engagement member 16K as an example) to each other when the interlocking swinging angle is zero is indicated as a virtual line V23. Moreover, the line that connects the driven axis 7J of the driven shaft member 7 and the rigidity adjustment axis 21DJ of the rigidity adjustment shaft member 21D to each other is indicated as a virtual reference line VX. When the spring swinging axis 16KJ is arranged at a position overlapping with the virtual reference line VX, the swinging angle of the femoral swinging arm 13 is zero and the interlocking swinging angle of the interlocking swinging member 16 is zero. In addition, the virtual tangential line VS and the virtual reference line VX are orthogonal to each other. Moreover, the angles formed between the virtual tangential line VS and the virtual line V23, i.e., an angle φa in FIG. 10 and an angle φb in FIG. 11 are rigidity adjustment angles.

FIG. 10 shows an example of a case in which the electric motor 21 is controlled such that the rigidity adjustment angle 4a becomes an almost right angle. Here, it is assumed that the femoral swinging arm 13 swings in a direction indicated by symbol L10 from a state in which the swinging angle is zero and that the interlocking swinging member 16 swings at an interlocking swinging angle of θR10 in a direction indicated by symbol R10 from the state in which the interlocking swinging angle is zero. In this case, the spring engagement member 16K moves from the position at which the spring engagement member 16K is coaxial with the rigidity adjustment shaft member 21D to the position of a spring engagement member 16K′ at which the spring engagement member 16K rotates rightward at the interlocking swinging angle of θR10. Thus, the expansion/contraction spring 23K is put in the state of an expansion/contraction spring 23K′ and expanded by ΔLR10. An urging force generated by the expansion of the expansion/contraction spring 23K′ turns into a force for swinging the interlocking swinging member 16 in the direction in which the interlocking swinging angle becomes zero.

In contrast to FIG. 10, FIG. 11 shows an example of a case in which the electric motor 21 is controlled such that the rigidity adjustment angle φb becomes about 45°. Here, as is the case with FIG. 10, it is assumed that the femoral swinging arm swings in the direction indicated by symbol L10 from the state in which the swinging angle is zero and that the interlocking swinging member 16 swings at the interlocking swinging angle of θR10 in the direction indicated by symbol R10 from the state in which the interlocking swinging angle is zero. In this case, the expansion/contraction spring 23K is put in the state of the expansion/contraction spring 23K′ and expanded by ΔLR11. However, although the interlocking swinging member 16 swings at the interlocking swinging angle of θR10 as is the case with FIG. 10, the expanded amount ΔLR11 is larger than the expanded amount ΔLR10 shown in FIG. 10. That is, an urging force of the expansion/contraction spring 23K′ in FIG. 10 is larger than an urging force of the expansion/contraction spring 23K′ in FIG. 11.

As described above, even though the interlocking swinging angle is the same, an expansion/contraction amount of the expansion/contraction spring 23K may be changed by changing the rigidity adjustment angle. In other words, an apparent spring constant of the expansion/contraction spring 23K seen from the interlocking swinging member 16 may be changed by changing the rigidity adjustment angle. That is, rigidity about the driving shaft member 6 may be adjusted by adjusting the rigidity adjustment angle. Note that the apparent spring constant of the expansion/contraction spring 23K seen from the interlocking swinging member 16 becomes the smallest when the rigidity adjustment angle is a right angle and becomes the largest when the rigidity adjustment angle is zero (0°≦rigidity adjustment angle≦90°.

The spring unit 23, the rigidity adjustment shaft member 21D, and the electric motor 21 (the rigidity adjustment shaft pivoting portion) described above constitute an apparent spring constant variable portion. The apparent spring constant variable portion varies the apparent spring constant of the expansion/contraction spring 23K seen from the interlocking swinging member 16 and varies the rigidity about the driving shaft member 6. In addition, the apparent spring constant variable portion, the driven shaft member 7, and the interlocking swinging member 16 constitute a rigidity variable portion. As described above, the “rigidity” indicates a torque per unit angle change required to swing the femoral swinging arm 13, the apparent spring constant of the expansion/contraction spring 23K seen from the interlocking swinging member 16 is related to the torque. Therefore, “the rigidity of the elastic body seen from the interlocking swinging member 16” includes “the apparent spring constant of the expansion/contraction spring 23K seen from the interlocking swinging member 16”. The spring constant represents a sort of rigidity. The rigidity of an elastic body may be varied to optimally reserve energy and to optimally output energy reserved. Furthermore, “the rigidity variable portion that varies rigidity of the elastic body seen from the interlocking swinging member 16” includes “the apparent spring constant variable portion that varies an apparent spring constant of the expansion/contraction spring 23K seen from the interlocking swinging member 16”.

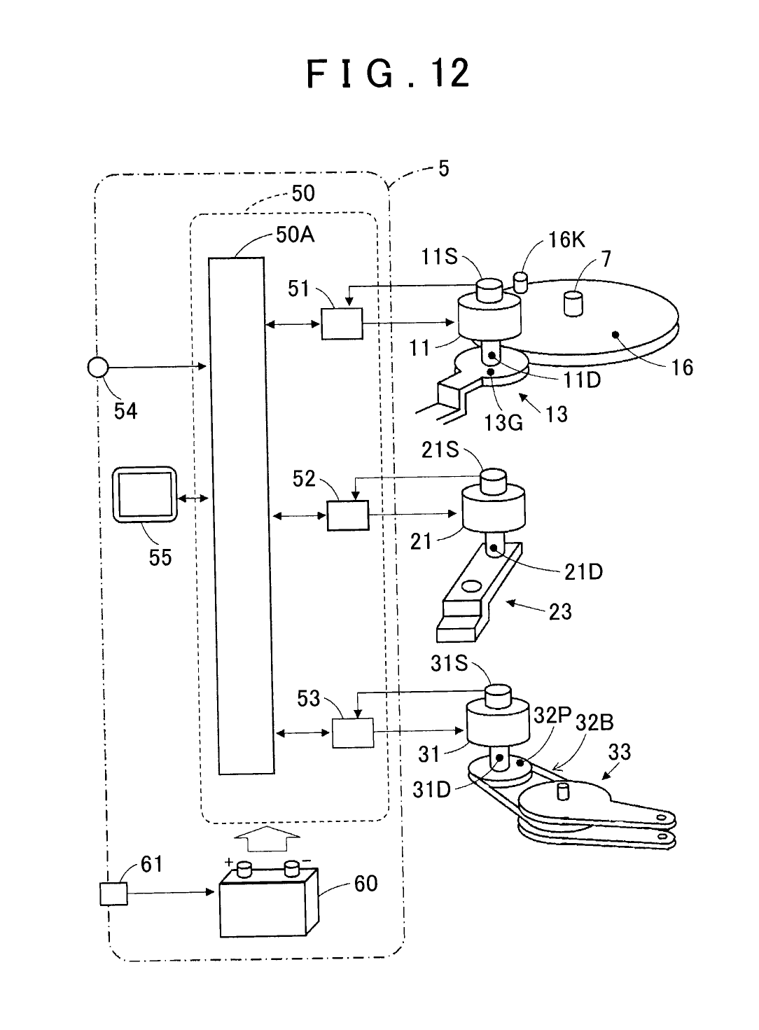

Next, a description will be given of the input/output of a control portion 50 with reference to FIG. 12. The control unit 5 accommodates the control portion 50 and a battery 60. In addition, the control unit 5 has a start switch 54, a touch panel 55 serving as an input/output portion, a connector 61 for charging the battery 60, or the like. Moreover, the control portion 50 (the control unit) has a central processing unit (CPU) 50A, motor drivers 51, 52, and 53, or the like. Note that although the control portion 50 also has a storage unit that stores a program for running the processing of the control portion 50, various measurement results, or the like, the storage unit is not shown in the figure.

As will be described later, the control portion 50 calculates a target swinging cycle and a target swinging angle to swing the femoral swinging arm 13 and outputs a driving signal to the electric motor 11 via the motor driver 51. Based on the driving signal from the control portion 50, the electric motor 11 swings the femoral swinging arm 13 at a prescribed cycle and a prescribed angle via the speed reducer 11D. In addition, a rotation speed and a rotation amount of the shaft of the electric motor 11 are detected by the rotation angle detection portion 11S, and a detection signal is input to the CPU 50A via the motor driver 51 while being input to the motor driver 51. The CPU 50A performs feedback control such that an actual swinging cycle and an actual swinging angle based on the detection signal from the rotation angle detection portion 11S get closer to the target swinging cycle and the target swinging angle.

In addition, as will be described later, the control portion 50 calculates a target rigidity adjustment angle of the spring unit 23 such that an apparent spring constant seen from the interlocking swinging member 16 has an optimum value, and outputs a driving signal to the electric motor 21 via the motor driver 52. Based on the driving signal from the control portion 50, the electric motor 21 pivots the spring unit 23 via the rigidity adjustment shaft member 21D. In addition, a rotation speed and a rotation amount of the shaft of the electric motor 21 are detected by the rotation angle detection portion 21S, and a detection signal is input to the CPU 50A via the motor driver 52 while being input to the motor driver 52. The CPU 50A performs feedback control such that an actual pivoting angle based on the detection signal from the rotation angle detection portion 21S gets closer to the target rigidity adjustment angle.

As will be described later, the control portion 50 calculates a target swinging cycle and a target swinging angle to swing the crus swinging arm 33 and outputs a driving signal to the electric motor 31 via the motor driver 53. Based on the driving signal from the control portion 50, the electric motor 31 swings the crus swinging arm 33 at a prescribed cycle and a prescribed angle via the speed reducer 31D, the pulley 32P, and the belt 32B. In addition, a rotation speed and a rotation amount of the shaft of the electric motor 31 are detected by the rotation angle detection portion 31S, and a detection signal is input to the CPU 50A via the motor driver 53 while being input to the motor driver 53. The CPU 50A performs feedback control such that an actual swinging cycle and an actual swinging angle based on the detection signal from the rotation angle detection portion 31S get closer to the target swinging cycle and the target swinging angle.

The start switch 54 is a switch for starting the control portion 50. In addition, the touch panel 55 is a device for inputting a user's height, weight, or the like and performing the display of a setting state or the like. Moreover, the connector 61 for charging is a connector to which a charging cable is connected to charge the battery 60.

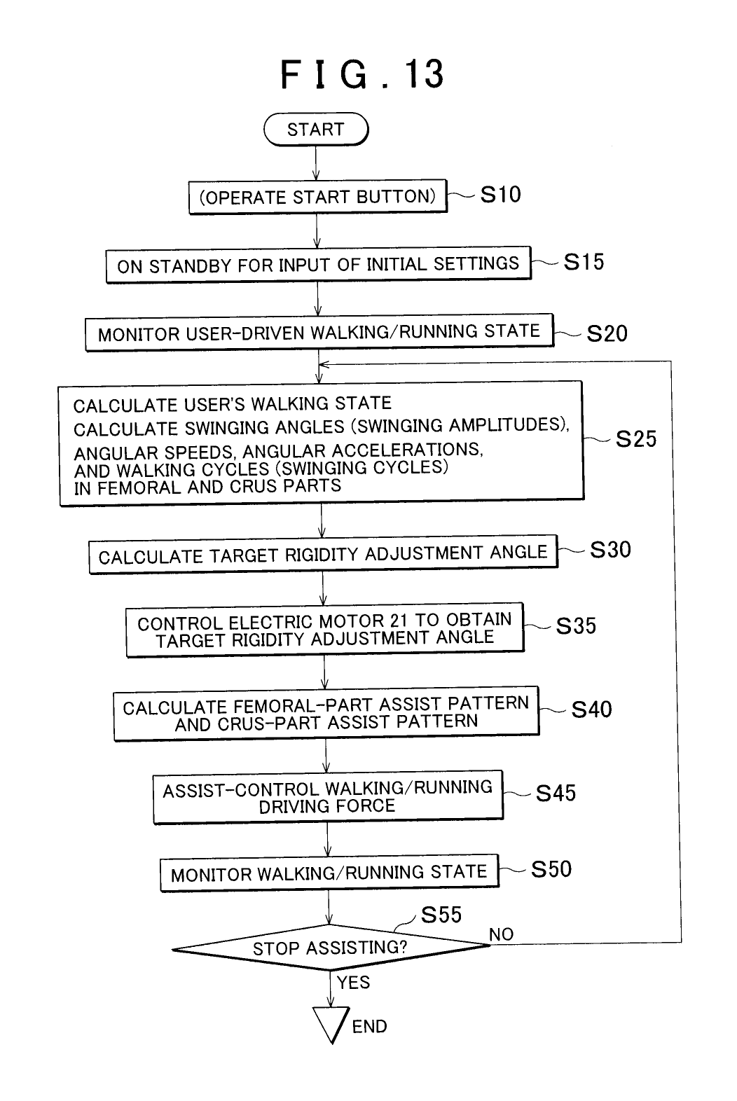

Next, a description will be given of the processing procedure of the control portion 50 with reference to a flowchart shown in FIG. 13. When a user operates the start button of the control unit (step S10), the control portion proceeds to step S15.

In step S15, the control portion is on standby for the input of user's initial settings via the touch panel. After confirming the input of a user's height and weight, the control portion proceeds to step S20. Note that when the user's input is not confirmed even after the elapse of a prescribed time, the control portion sets, for example, a default standard height and weight and proceeds to step S20.

In step S20, the control portion measures a user's waking (or running) state without energizing the electric motors 11, 21, and 31 for a prescribed period and stores detection signals from the rotation angle detection portions 11S and 31S in the storage unit as measurement data so as to correspond to a measurement time. The shafts of the electric motors 11 and 31 are configured to idle at a non-energizing time. Note that the shaft of the electric motor 21 is configured to be locked without idling at the non-energizing time. Then, after collecting the measurement data for, for example, a prescribed number of steps or a prescribed time, the control portion proceeds to step S25.

In step S25, the control portion calculates a swinging angle (or a swinging amplitude) of the femoral swinging arm from the measurement data based on the detection signal from the rotation angle detection portion 11S and calculates a walking cycle (or a swinging cycle) from an angular speed and an angular acceleration of the femoral swinging arm. In addition, the control portion similarly calculates a swinging angle (or a swinging amplitude) of the crus swinging arm from the measurement data based on the detection signal from the rotation angle detection portion 31S and calculates a walking cycle (or a swinging cycle) from an angular speed and an angular acceleration of the crus swinging arm. Then, the control portion proceeds to step S30.

In step S30, the control portion calculates a target rigidity adjustment angle as optimum joint rigidity based on the swinging angle and the swinging cycle of the femoral swinging arm calculated in step S25 and the user's height and weight or the like input in step S15. After that, the control portion proceeds to step S35. Note that a method for calculating the target rigidity adjustment angle will be described in detail later.

In step S35, the control portion controls the electric motor 21 to set a rigidity adjustment angle of the spring unit 23 (the pivoting member 23A) at the target rigidity adjustment angle calculated in step S30. After that, the control portion proceeds to step S40.

In step S40, the control portion calculates the pattern of assisting the femoral part of a user (the pattern of outputting a driving signal to the electric motor 11, or the like) and the pattern of assisting the crus part of the user (the pattern of outputting a driving signal to the electric motor 31) based on the swinging angle and the swinging cycle of the femoral swinging arm and the swinging angle and the swinging cycle of the crus swinging arm calculated in step S25, an output voltage of the battery, or the like. After that, the control portion proceeds to step S45.

In step S45, the control portion starts outputting driving signals to the electric motors 11 and 31 based on the patterns calculated in step S40 to swing the femoral swinging arm 13 and the crus swinging arm 33 and assists the user's walking (or running) action so as to continue the user's walking (or running) action. After that, the control portion proceeds to step S50. Note that the output of the driving signals to the electric motors 11 and 31 is continued even after the control portion transits to other steps.

In step S50, the control portion stores, as in the measurement of step S20, detection signals from the rotation angle detection portions 11S and 31S in the storage unit as measurement data so as to correspond to a measurement time while operating the electric motors 11 and 31 and assisting the user's walking (or running) action. After that, the control portion proceeds to step S55. Note that the collection of the measurement data is continued even after the control portion transits to other steps.

In step S55, the control portion determines whether the user wants to stop assisting the walking (or running) action based on the measurement data collected in step S50. When determining that the user wants to stop assisting the walking (or running) action (Yes), the control portion stop outputting the driving signals to the electric motors 11 and 31 to end the processing. On the other hand, when determining that the user does not want to stop assisting the walking (or running) action (No), the control portion returns to step S25.

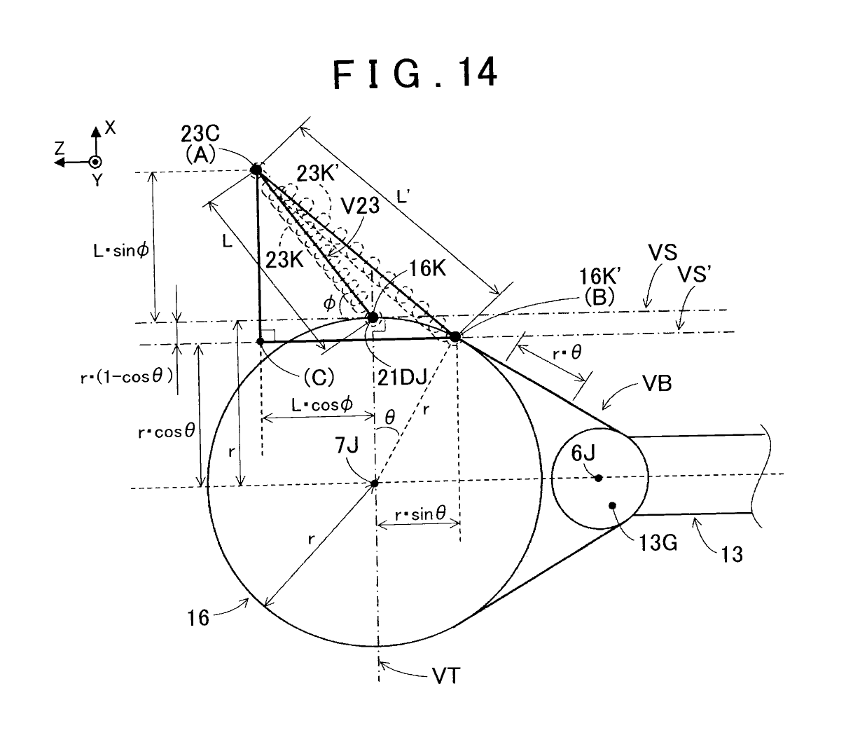

Next, a description will be given, with reference to FIG. 14, of a procedure for calculating the target rigidity adjustment angle performed in step S30 of the flowchart shown in FIG. 13. FIG. 14 is a view schematically showing the femoral swinging arm 13, the interlocking swinging member 16, the spring engagement member 16K, the swinging following shaft member 23C, and the expansion/contraction spring 23K. Note that the swinging motion of the femoral swinging arm 13 in an example shown in FIG. 14 is configured to be transmitted to the interlocking swinging member 16 via a belt VB.

In FIG. 14, a tangential line contacting the rigidity adjustment axis 21DJ set on the periphery of the interlocking swinging member 16 is indicated as a virtual tangential line VS. In addition, a line passing through the rigidity adjustment axis 21DJ and the driven axis 7J is indicated as a virtual line VT. Moreover, the interlocking swinging member 16 is indicated as a perfect circle having a radius r about the driven axis 7J. Further, one end (a portion corresponding to the one end) of the expansion/contraction spring 23K engages with the swinging following shaft member 23C (exemplifying the spring fixing end) of the spring unit, and the other end (a portion corresponding to the other end) thereof engages with the spring engagement member 16K (exemplifying the spring swinging end). Furthermore, the expansion/contraction spring 23K has a free length when the spring engagement member 16K is coaxial with the rigidity adjustment axis 21DJ, and the free length is indicated as L. Furthermore, when the interlocking swinging angle of the interlocking swinging member 16 is zero, the spring engagement member 16K is coaxial with the rigidity adjustment axis 21DJ.

When the interlocking swinging member 16 swings clockwise at an angle of θ in an interlocking manner from the state in which the interlocking swinging angle of the interlocking swinging member 16 is zero (the state in which the spring engagement member 16K is coaxial with the rigidity adjustment axis 21DJ), the spring engagement member 16K moves from the position at which the spring engagement member 16K is coaxial with the rigidity adjustment axis 21DJ to a position indicated by symbol 16K′, whereby the expansion/contraction spring 23K is put in a position and an expansion/contraction state indicated by symbol 23K′. Here, the length of the expansion/contraction spring indicated by symbol 23K′ is indicated as L′. A line passing through symbol 16K′ and parallel to the virtual tangential line VS is indicated as a virtual line VS′. In addition, the position of the swinging following shaft member 23C is set as a position (A), the position of the intersection between the virtual line VS' and a perpendicular line dropping from the position (A) to the virtual line VS' is set as a position (C), and the position of symbol 16K′ is set as a position (B). Moreover, the rigidity adjustment angle, i.e., the angle formed between the virtual tangential line VS and the virtual line V23 connecting the swinging following shaft member 23C and the rigidity adjustment axis 21DJ to each other is indicated as an angle φ.

According to the above respective settings, the distance between the driven axis 7J and symbol 16K′ is indicated as r. In addition, the distance between the driven axis 7J and the virtual tangential line VS is indicated as r. Moreover, the distance between the position (B) and the virtual line VT is indicated as r·sin θ. Further, the distance between the position (C) and the virtual line VT is indicated as L·cos φ. Furthermore, the distance between the virtual tangential line VS and the virtual line VS' is indicated as r−r·cos θ=r·(1−cos θ). Furthermore, the distance between the position (A) and the virtual tangential line VS is indicated as L·sin φ. Furthermore, when the angle θ, i.e., the interlocking swinging angle of the interlocking swinging member 16 is a substantially slight angle, a displacement amount in the peripheral direction of the interlocking swinging member 16 that represents a movement length of the belt VB is indicated as r·θ.

Here, when a user's walking frequency is indicated as f and an angular speed at this time is indicated as ω, the following formula (1) is established. The walking frequency f may be calculated from a measured user's walking (or running) cycle. Accordingly, a value ω in the following formula (1) may be calculated.

ω=2·π·f

In addition, a spring constant when the expansion/contraction spring 23K expands/contracts in the direction of the free length is indicated as k, and an apparent spring constant of the expansion/contraction spring seen from the interlocking swinging member 16 when the rigidity adjustment angle is an angle φ is indicated as k′. Moreover, inertia moment about the driven axis 7J including a user's lower limb, the femoral swinging arm 13, and the interlocking swinging member 16 is indicated as I. For example, it is possible to calculate the inertia moment I from the (established) total mass of the respective members swinging about the driven axis 7J, the (established) gravity position of the total mass, and the mass of a lower limb and a gravity position estimated from a user's weight and height, and the following formula (2) is established. Since the value of co and the inertia moment I are calculated in the above manner, the apparent spring constant k′ may be calculated from the following formula (2).

ω=√(k′/I)

k′=I·ω²

Further, the following formula (3) is established according to the energy conservation law. Since L, r, θ, k, and k′ are calculated in the above manner, L′ may be calculated by the following formula (3).

(1/2)·k′·(r·θ)²=(1/2)·k·(L′−L)²

L′=L+r·θ·√(k′/k)

Furthermore, in FIG. 14, a triangle having apexes at the positions (A), (B), and (C) is a right-angle triangle. Therefore, the following formula (4) is established according to the Pythagorean theorem.

(r·sin θ+L·cos φ)²+[r·(1−cos θ)+L·sin φ]²=L′²

When the above formula (4) is organized, the following formula (5) may be obtained.

cos [(θ/2)−φ]=[L′²−L²−2·r²·(1−cos θ)]/4·L·r·sin(θ/2)

Here, when the above formula (5) is replaced by [L′²−L²−2·r²·(1−cos θ)]/4·L·r·sin(θ/2)=χ, the following formula (6) may be obtained since χ=√(k′/k) is established where θ=0. Since L′, L, r, θ, k, and k′ are calculated in the above manner, it is possible to calculate χ. As a result, an angle φ may be calculated. The calculated angle φ is a target rigidity adjustment angle.

φ=(θ/2)+cos⁻¹χ where φ>θ/2

φ=(θ/2)−cos⁻¹χ where φ≦θ/2

As described above, based on the swinging frequency (f) of the femoral swinging arm 13 about the driving shaft member 6, the inertia moment (I) about the driving shaft member 6 in a swinging object including the femoral swinging arm 13, the spring constant (k) of the expansion/contraction spring 23K, the free length (L) of the expansion/contraction spring 23K, the distance (r) between the driven shaft member 7 and the rigidity adjustment shaft member, and the interlocking swinging angle (the angle θ), the rigidity adjustment angle (the angle φ) is adjusted using the control portion 50 such that the resonance point of the expansion/contraction spring coincides with the swinging frequency of the swinging object.

Thus, the rigidity adjustment angle φ is set such that the resonance point of the expansion/contraction spring 23K coincides with the swinging frequency of a swinging object (the whole object swinging about the driving shaft member 6) including the swinging arm 13 to establish the energy conservation law, whereby power consumed by the electric motor 11 may be minimized. Note that the rigidity adjustment angle may not be calculated according to the above formula but may be calculated according to other methods. That is, the rigidity adjustment angle is minutely changed, and the consumption power of the electric motor 11 for a prescribed cycle is measured at the rigidity adjustment angle. After that, the rigidity adjustment angle is minutely changed again, and the consumption power of the electric motor 11 for a prescribed cycle is measured. By repeatedly measuring the consumption power of the electric motor 11 in this manner, the rigidity adjustment angle resulting in the minimum consumption power may be calculated.

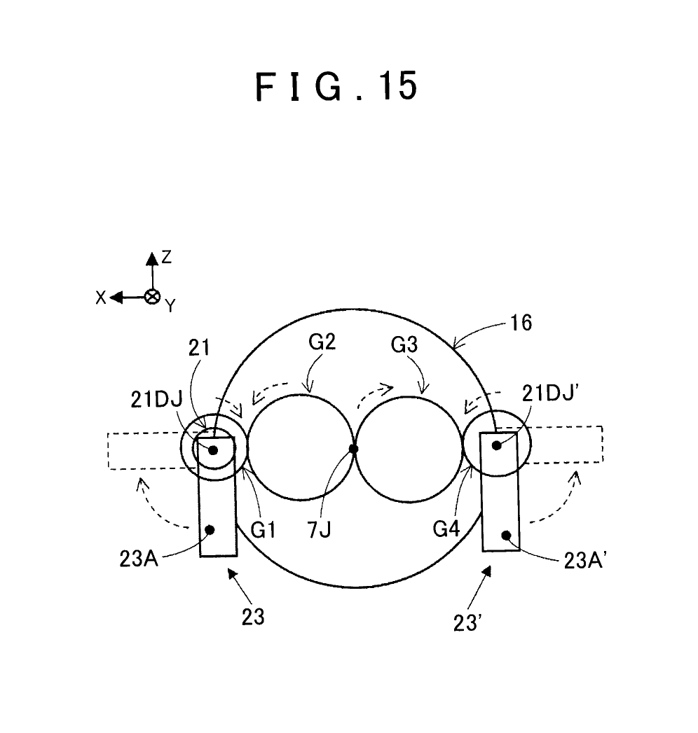

In a case in which the expansion/contraction spring of the spring unit is effective only in its expansion direction but is not effective in its contraction direction (for example, a case in which the expansion/contraction spring is put in a state shown in the schematic diagram of FIG. 10), or in a case in which the expansion/contraction spring of the spring unit shown in FIG. 5 is effective in both expansion and contraction directions but an urging force for the interlocking swinging angle is not sufficient, or the like, the interlocking swinging member 16 may have two spring units, i.e., the spring unit 23 and a spring unit 23′ as shown in the example of FIG. 15. Note that a portion corresponding to the other end of the expansion/contraction spring of the spring unit 23 is connected to (engages with) a spring engagement member (not shown) arranged near the rigidity adjustment axis 21DJ and a portion corresponding to the other end of the expansion/contraction spring of the spring unit 23′ is connected to (engages with) a spring engagement member (not shown) arranged near a rigidity adjustment axis 21DJ′.

In this case, the pivoting member 23A (pivoting about the rigidity adjustment axis 21DJ) of the spring unit 23 is pivoted and driven by the electric motor 21. In addition, a pivoting member 23A′ (pivoting about the rigidity adjustment axis 21DJ′) of the spring unit 23′ receives a pivoting driving force via a gear G1 attached to the pivoting member 23A, gears G2 and G3 supported by the bracket 22 (see FIG. 1), and a gear G4 attached to the pivoting member 23A′. By appropriately setting the gear ratios between the adjacent gears, the rigidity adjustment angle of the spring unit 23 and the rigidity adjustment angle of the spring unit 23′ may coincide with each other.

For example, even when the expansion/contraction springs are springs that generate an urging force only in their expansion directions (see FIGS. 10 and 11), the expansion/contraction spring of one apparent spring constant variable portion may be configured to expand in its expansion direction relative to swinging motion in one direction and the expansion/contraction spring of the other apparent spring constant variable portion may be configured to expand in its expansion direction relative to swinging motion in the other direction. Therefore, the spring unit having a complicated structure shown in FIG. 5 is not required. Accordingly, the structure of the spring unit may be further simplified.

The swinging joint device 1 of the first embodiment described above is used for the left leg of a user. However, the control unit 5 may assist the walking (or running) actions of both legs of a user with the addition of a base portion for the right leg (symmetrical to the base portion 2), a femoral swinging portion for the right leg (symmetrical to the respective members indicated by symbols 11, 12, 13, and 19), a rigidity adjustment portion for the right leg (symmetrical to the respective members indicated by symbols 16, 21, 22, and 23), and a crus swinging portion for the right leg (symmetrical to the respective members indicated by symbols 31, 32, 32P, 32B, 33, 34, 35, 36, and 39).

The swinging joint device of a second embodiment is one in which the electric motor 11 (and the rotation angle detection portion 11S) are removed from the swinging joint device 1 of the first embodiment shown in FIGS. 1 to 4 and a rotation angle detection portion capable of detecting a swinging angle of the femoral swinging arm 13 is added to the swinging joint device 1. In the second embodiment, the motion of the femoral part may not be assisted by an electric motor when a user walks (or runs), but the motion of the crus part may be assisted by the electric motor 31. In addition, since the swinging joint device has the rigidity adjustment unit, it may set the rigidity adjustment angle at an appropriate angle according to the energy conservation law to appropriately reduce a momentum of the femoral part of a user.

In addition, as is the case with the first embodiment, the control unit 5 may assist the walking (or running) action of both legs of a user with the addition of a base portion for the right leg (symmetrical to the base portion 2), a femoral swinging portion for the right leg (symmetrical to the respective members indicated by symbols 11, 12, 13, and 19), a rigidity adjustment portion for the right leg (symmetrical to the respective members indicated by symbols 16, 21, 22, and 23), and a crus swinging portion for the right leg (symmetrical to the respective members indicated by symbols 31, 32, 32P, 32B, 33, 34, 35, 36, and 39).

The swinging joint device of a third embodiment is one in which the electric motor 31, the bracket 32, the pulley 32P, the belt 32B, the crus swinging arm 33, the crus relaying arm 34, the crus arm 35, the foot holding portion 36, and the crus attachment portion 39 are removed from the swinging joint device 1 of the first embodiment shown in FIGS. 1 to 4. In the third embodiment, the motion of the femoral part is assisted by the electric motor 11 when a user walks (or runs), but the motion of the crus part is not assisted. Note that since the swinging joint device has the rigidity adjustment unit, it may set the rigidity adjustment angle at an appropriate angle according to the energy conservation law to further reduce the consumption power of the electric motor 11.

In addition, as is the case with the first embodiment, the control unit 5 may assist the walking (or running) action of both legs of a user with the addition of a base portion for the right leg (symmetrical to the base portion 2), a femoral swinging portion for the right leg (symmetrical to the respective members indicated by symbols 11, 12, 13, and 19), and a rigidity adjustment portion for the right leg (symmetrical to the respective members indicated by symbols 16, 21, 22, and 23).

The swinging joint device of a fourth embodiment is one in which the electric motor 11 (and the rotation angle detection portion 11S) are removed from the swinging joint device of the third embodiment and a rotation angle detection portion capable of detecting a swinging angle of the femoral swinging arm 13 is added to the swinging joint device. In the fourth embodiment, the motion of the crus part may not be assisted when a user walks (or runs). In addition, the motion of the femoral part of a user may not be assisted. However, since the swinging joint device has the rigidity adjustment unit, it may set the rigidity adjustment angle at an appropriate angle according to the energy conservation law to appropriately reduce a momentum of the femoral part of a user.

In addition, as is the case with the first embodiment, the control unit 5 may assist the walking (or running) action of both legs of a user with the addition of a base portion for the right leg (symmetrical to the base portion 2), a femoral swinging portion for the right leg (symmetrical to the respective members indicated by symbols 11, 12, 13, and 19), and a rigidity adjustment portion for the right leg (symmetrical to the respective members indicated by symbols 16, 21, 22, and 23).

The structure, the configuration, the shape, the appearance, and The method for controlling rigidity of a swinging joint of the swinging joint device of the invention may be changed, added, or deleted in various ways without departing from the scope of the invention.

The application of the swinging joint device and the walking-ability assisting device described in the embodiment is not limited to assisting the swinging motion (walking or running) of the lower limb of a user, and the application of the method for controlling the rigidity of a swinging joint described in the embodiment is not limited to assisting the swinging motion of the lower limb of a user. However, the swinging joint device or the walking-ability assisting device and the method for controlling the rigidity of a swinging joint may be applied to various objects that perform cyclic swinging motion.

In the embodiment, the swinging motion of the femoral swinging arm 13 is transmitted to the interlocking swinging member 16 by the gears. However, the power transmission portion may be constituted by a belt, a pulley, a link mechanism, or the like besides the gears. Similarly, the swinging rotation motion of the electric motor 31 is transmitted to the crus swinging arm 33 by the pulley and the belt but may be transmitted by gears, a link mechanism, or the like besides the belt and the pulley. In addition, in the example of FIG. 15, the pivoting driving force is transmitted by a pivoting member driving force transmission portion using the gears. However, the pivoting driving force may be transmitted via a pulley, a belt, a link mechanism, or the like besides the gears.

Moreover, the example in which the expansion/contraction spring 23K is used as the elastic body is explained in the embodiment but various elastic bodies may be used instead of the expansion/contraction spring 23K. For example, a spirally-wound spring is used as the expansion/contraction spring of the embodiment, but other springs such as a plate spring and a wave spring may be used. An elastic body made of elastomer such as rubber and a resin, liquid such as oil, or gas may be used. The elastic body may be changed according to a momentum of an object (action) whose energy is to be reserved or a reserved energy amount. When an energy amount to be reserved is relatively small, it is effective to use elastomer that reserves relatively less energy. Further, for a user's action such as walking and running, it is effective to use the expansion/contraction spring because of a relatively large reserved amount of energy, a degree of a spring constant (rigidity), and easiness of adjustment (for example, in a case of a spring in the shape of a coil, the number of turns of the spring, the thickness of a wire, or the like) and so on. For this reason, it is effective to use the expansion/contraction spring. Furthermore, the expansion/contraction spring is superior in terms of cost.

What is claimed is:

1. A swinging joint device comprising:
a driving shaft member;
a first swinging arm that is swingably supported about the driving shaft member;
a driven shaft member that is arranged parallel to the driving shaft member;
an interlocking swinging member that is connected to the first swinging arm via a power transmission portion to swing about the driven shaft member in an interlocking manner with swinging of the first swinging arm while swinging at an interlocking swinging angle smaller than a first swinging angle that is a swinging angle of the first swinging arm;
an elastic body that is connected to the interlocking swinging member to generate an urging force corresponding to the interlocking swinging angle, the urging force being generated in a direction opposite to an interlocking swinging direction of the interlocking swinging member;
a rigidity variable portion that varies rigidity of the elastic body seen from the interlocking swinging member;
a first angle detection portion that detects one of the first swinging angle and the interlocking swinging angle; and
a control portion that controls the rigidity variable portion according to one of the first swinging angle and the interlocking swinging angle detected by the first angle detection portion to adjust the rigidity of the elastic body seen from the interlocking swinging member.

2. The swinging joint device according to claim 1, wherein
the elastic body is an expansion/contraction spring, and
the rigidity variable portion is an apparent spring constant variable portion that varies an apparent spring constant of the expansion/contraction spring seen from the interlocking swinging member.

3. The swinging joint device according to claim 2, wherein
the apparent spring constant variable portion is constituted by a rigidity adjustment shaft member that is arranged at a position near a periphery of the interlocking swinging member and arranged parallel to the driven shaft member, a rigidity adjustment shaft pivoting portion that pivots the rigidity adjustment shaft member, a pivoting member that is connected to the rigidity adjustment shaft member to pivot with the rigidity adjustment shaft member, and the expansion/contraction spring,
a portion corresponding to a first end of the expansion/contraction spring is connected to a spring fixing end of the pivoting member that is at a position away from the rigidity adjustment shaft member,
a portion corresponding to a second end of the expansion/contraction spring is connected to a spring swinging end that is at a position near the periphery of the interlocking swinging member, the spring swinging end being coaxial with the rigidity adjustment shaft member at the position when the interlocking swinging angle is zero,
the expansion/contraction spring connected to the spring fixing end and the spring swinging end has a free length when the interlocking swinging angle is zero, and
the control portion adjusts a rigidity adjustment angle according to the interlocking swinging angle to adjust the apparent spring constant of the expansion/contraction spring seen from the interlocking swinging member, the rigidity adjustment angle being an angle formed between a virtual tangential line and a virtual line, the virtual tangential line representing a tangential line that is set on a circumference of a virtual interlocking swinging circle serving as a circle having a distance between the driven shaft member and the rigidity adjustment shaft member as a radius about the driven shaft member and that is set at a position of the rigidity adjustment shaft member, the virtual line connecting the spring swinging end and the spring fixing end to each other when the interlocking swinging angle is zero.

4. The swinging joint device according to claim 3, wherein
two apparent spring constant variable portions are attached to the interlocking swinging member as the apparent spring constant variable portion.

5. The swinging joint device according to claim 4, wherein
a first one of the two apparent spring constant variable portions attached to the interlocking swinging member has the rigidity adjustment shaft pivoting portion, and
a second one of the two apparent spring constant variable portions attached to the interlocking swinging member does not have the rigidity adjustment shaft pivoting portion but has a pivoting member power transmission portion that transmits, to the pivoting member of the second apparent spring constant variable portion, a pivoting driving force of the pivoting member of the first apparent spring constant variable portion generated by the rigidity adjustment shaft pivoting portion of the first apparent spring constant variable portion

6. The swinging joint device according to claim 1, further comprising:
a first driving portion that swings the first swinging arm about the driving shaft member based on a control signal from the control portion.

7. The swinging joint device according to claim 1, further comprising:
a second swinging arm that is swingably supported about the driving shaft member;
a second angle detection portion that detects a second swinging angle as a swinging angle of the second swinging arm;
a second driving portion that swings the second swinging arm about the driving shaft member based on a control signal from the control portion; and
a swinging link member that is connected to the first swinging arm and the second swinging arm to operate based on the first swinging angle of the first swinging arm and the second swinging angle of the second swinging arm.

8. The swinging joint device according to claim 1, wherein
the power transmission portion that transmits swinging of the first swinging arm to the interlocking swinging member is constituted by one of a gear, a belt, and a link mechanism.

9. A walking-ability assisting device for applying an assisting force to motion of a lower limb, the walking-ability assisting device comprising:
a waist-side attachment portion that is attached to a waist-side part;
a first swinging arm that is arranged on a lateral side of a femur and has a shaft hole near an upper end thereof;
a femoral attachment portion that is attached to the first swinging arm and put on the femur;
a driving shaft member that is inserted into the shaft hole of the first swinging arm to swingably support the first swinging arm back and forth relative to the waist-side attachment portion;
a rigidity variable portion that varies rigidity about the driving shaft member; and
a control portion that controls the rigidity about the driving shaft member varied by the rigidity variable portion.

10. The walking-ability assisting device according to claim 9, wherein
the rigidity variable portion has an expansion/contraction spring,
the expansion/contraction spring has a free length when a swinging angle of the first swinging arm is zero, and
an expansion/contraction amount of the expansion/contraction spring is varied relative to the swinging angle of the first swinging arm to vary the rigidity about the driving shaft member.

11. The walking-ability assisting device according to claim 10, wherein
the rigidity variable portion is constituted by a driven shaft member that is arranged parallel to the driving shaft member, an interlocking swinging member that is swingably supported about the driven shaft member and connected to the first swinging arm via a power transmission portion to swing in an interlocking manner with swinging of the first swinging arm while swinging at an interlocking swinging angle smaller than a swinging angle of the first swinging arm, a rigidity adjustment shaft member that is arranged at a position near a periphery of the interlocking swinging member and arranged parallel to the driven shaft member, a rigidity adjustment shaft pivoting portion that pivots the rigidity adjustment shaft member, a pivoting member that is connected to the rigidity adjustment shaft member to pivot with the rigidity adjustment shaft member, and the expansion/contraction spring,
a portion corresponding to a first end of the expansion/contraction spring is connected to a spring fixing end of the pivoting member that is at a position away from the rigidity adjustment shaft member,
a portion corresponding to a second end of the expansion/contraction spring is connected to a spring swinging end that is at a position near the periphery of the interlocking swinging member, the spring swinging end being coaxial with the rigidity adjustment shaft member at the position when the interlocking swinging angle is zero,
the expansion/contraction spring connected to the spring fixing end and the spring swinging end has a free length when the interlocking swinging angle is zero, and
the control portion controls the rigidity adjustment shaft pivoting portion to adjust a rigidity adjustment angle according to the interlocking swinging angle to adjust the apparent spring constant of the expansion/contraction spring seen from the interlocking swinging member, the rigidity adjustment angle being an angle formed between a virtual tangential line and a virtual line, the virtual tangential line representing a tangential line that is set on a circumference of a virtual interlocking swinging circle serving as a circle having a distance between the driven shaft member and the rigidity adjustment shaft member as a radius about the driven shaft member and that is set at a position of the rigidity adjustment shaft member, the virtual line connecting the spring swinging end and the spring fixing end to each other when the interlocking swinging angle is zero.

12. The walking-ability assisting device according to claim 11, wherein
the control portion adjusts the rigidity adjustment angle such that a resonance point of the expansion/contraction spring coincides with a swinging frequency of a swinging object including the first swinging arm, based on a swinging frequency of the first swinging arm about the driving shaft member, inertia moment about the driving shaft member in the swinging object, a spring constant of the expansion/contraction spring, the free length of the expansion/contraction spring, a distance between the driven shaft member and the rigidity adjustment shaft member, and the interlocking swinging angle.

13. The walking-ability assisting device according to claim 9, further comprising:
a first driving portion that swings the first swinging arm about the driving shaft member based on a control signal from the control portion.

14. The walking-ability assisting device according to claim 9, further comprising:
a second swinging arm that is swingably supported about the driving shaft member;
a second angle detection portion that detects a second swinging angle as a swinging angle of the second swinging arm;
a second driving portion that swings the second swinging arm about the driving shaft member based on a control signal from the control portion; and
a swinging link member that is connected to the first swinging arm and the second swinging arm to operate based on the first swinging angle of the first swinging arm and the second swinging angle of the second swinging arm.

15. A method for controlling rigidity of a swinging joint, the swinging joint including a driving shaft member, a first swinging arm that is swingably supported about the driving shaft member, a driven shaft member that is arranged parallel to the driving shaft member, an interlocking swinging member that is connected to the first swinging arm via a power transmission portion to swing about the driven shaft member in an interlocking manner with swinging of the first swinging arm while swinging at an interlocking swinging angle smaller than a swinging angle of the first swinging arm, an elastic body that is connected to the interlocking swinging member to generate an urging force corresponding to the interlocking swinging angle, the urging force being generated in a direction opposite to an interlocking swinging direction of the interlocking swinging member, a rigidity variable portion that varies rigidity of the elastic body seen from the interlocking swinging member, and a control portion that controls the rigidity variable portion, the method comprising:
adjusting the rigidity of the elastic body seen from the interlocking swinging member according to the interlocking swinging angle using the control portion and the rigidity variable portion.

16. The method for controlling rigidity of a swinging joint according to claim 15, wherein
the elastic body is an expansion/contraction spring, and
the rigidity variable portion is an apparent spring constant variable portion that varies an apparent spring constant of the expansion/contraction spring seen from the interlocking swinging member.

17. The method for controlling rigidity of a swinging joint according to claim 16, wherein
the apparent spring constant variable portion is constituted by a rigidity adjustment shaft member that is arranged at a position near a periphery of the interlocking swinging member and arranged parallel to the driven shaft member, a rigidity adjustment shaft pivoting portion that pivots the rigidity adjustment shaft member, a pivoting member that is connected to the rigidity adjustment shaft member to pivot with the rigidity adjustment shaft member, and the expansion/contraction spring,
a portion corresponding to a first end of the expansion/contraction spring is connected to a spring fixing end of the pivoting member that is at a position away from the rigidity adjustment shaft member,
a portion corresponding to a second end of the expansion/contraction spring is connected to a spring swinging end that is at a position near the periphery of the interlocking swinging member, the spring swinging end being coaxial with the rigidity adjustment shaft member at the position when the interlocking swinging angle is zero,
the expansion/contraction spring connected to the spring fixing end and the spring swinging end has a free length when the interlocking swinging angle is zero, and
the rigidity adjustment shaft pivoting portion is controlled using the control portion to adjust a rigidity adjustment angle according to the interlocking swinging angle to adjust the apparent spring constant of the expansion/contraction spring seen from the interlocking swinging member, the rigidity adjustment angle being an angle formed between a virtual tangential line and a virtual line, the virtual tangential line representing a tangential line that is set on a circumference of a virtual interlocking swinging circle serving as a circle having a distance between the driven shaft member and the rigidity adjustment shaft member as a radius about the driven shaft member and that is set at a position of the rigidity adjustment shaft member, the virtual line connecting the spring swinging end and the spring fixing end to each other when the interlocking swinging angle is zero.

18. The method for controlling rigidity of a swinging joint according to claim 17, wherein
the rigidity adjustment angle is adjusted using the control portion such that a resonance point of the expansion/contraction spring coincides with a swinging frequency of a swinging object including the first swinging arm, based on a swinging frequency of the first swinging arm about the driving shaft member, inertia moment about the driving shaft member in the swinging object, a spring constant of the expansion/contraction spring, the free length of the expansion/contraction spring, a distance between the driven shaft member and the rigidity adjustment shaft member, and the interlocking swinging angle.
